(19) United States
(12) Patent Application Publication
(10) Pub. No.: US 2025/0120654 A1
(43) Pub. Date: Apr. 17, 2025

(54) IMPLANTABLE MEDICAL DEVICE WITH SENSING AND COMMUNICATION FUNCTIONALITY

(71) Applicant: CANARY MEDICAL SWITZERLAND AG, Baar (CH)

(72) Inventors: Kimberly Salant, Asbury, NJ (US); Mark A. Adler, Carlsbad, CA (US)

(21) Appl. No.: 18/684,633

(22) PCT Filed: Aug. 24, 2022
(86) PCT No.: PCT/US2022/041402

§ 371 Date: Feb. 16, 2024

Related U.S. Application Data
(60) Provisional application No. 63/236,599, filed on Aug. 24, 2021.

(51) Int. Cl. A61B 5/00; A61B 5/0205; A61B 5/07; A61B 17/00; A61B 17/12

(52) U.S. Cl. CPC A61B 5/6862; A61B 5/0205; A61B 5/076; A61B 17/12113; A61B 17/1214; A61B 2017/00867; A61B 2017/1205

(57) ABSTRACT

An implantable system for monitoring a vascular structure can include a sensor configured to be positioned within the vascular structure and further configured to detect a property of a flow of blood in the vascular structure, a communication circuitry configured to be positioned within the vascular structure, the communication circuitry connected to the sensor and configured to transmit data related to the property of the flow of blood detected by the electronic sensor, and an antenna configured to be positioned within the vascular structure, the antenna connected to the communication circuitry and further configured to facilitate transmission of the data related to the property of the flow of blood detected by the sensor.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### TECHNICAL FIELD

The present disclosure relates generally to medical devices with sensing and communication functionality, systems including such devices, methods of using such devices and systems and the data generated therefrom, and devices and methods to address problems associated with an implanted medical device with a sensor.

### BACKGROUND

After treating an internal injury or other internal defects, it can be difficult to monitor the progress of the patient's recovery. For example, aneurysms occur when the patient's artery wall weakens, which causes the weakened area to balloon. Aneurysms can occur throughout the body (e.g., the aorta, the brain, or elsewhere). A patient with an aneurysm will often experience no symptoms until the aneurysm ruptures. A ruptured aneurysm can result in internal bleeding, a stroke, and, occasionally, it can be fatal.

All of the subject matter discussed in the Background section is not necessarily prior art and should not be assumed to be prior art merely as a result of its discussion in the Background section. Along these lines, any recognition of problems in the prior art discussed in the Background section or associated with such subject matter should not be treated as prior art unless expressly stated to be prior art. Instead, the discussion of any subject matter in the Background section should be treated as part of the inventor's approach to the particular problem, which in and of itself may also be inventive.

### SUMMARY

This Brief Summary has been provided to introduce certain concepts in a simplified form that are further described in detail below in the Detailed Description. Except where otherwise expressly stated, this Brief Summary is not intended to identify key or essential features of the claimed subject matter, nor is it intended to limit the scope of the claimed subject matter.

The details of one or more embodiments are set forth in the description below. The features illustrated or described in connection with one example embodiment may be combined with the features of other embodiments. Thus, any of the various embodiments described herein can be combined to provide further embodiments. Aspects of the embodiments can be modified, if necessary to employ concepts of the various patents, applications and publications as identified herein to provide yet further embodiments. Other features, objects and advantages will be apparent from the description, the drawings, and the claims.

One method of treating an aneurysm is coil embolization. During this procedure, the physician implants a structure, such as a metal coil, into the aneurysm to close off the aneurysm and reduce the risk of bleeding. After the procedure, it is difficult to monitor the progress of the aneurysm, especially if the aneurysm is located in the brain. This results in multiple follow up visits with the doctor, which can increase the patient's medical care costs. Moreover, compactions can occur at the neck of the aneurysm (i.e., the transition between the parent artery and the aneurysm). Currently, there is no mechanism to reliably monitor the aneurysm after treatment.

Certain aspects of this disclosure are directed toward an implantable sensor assembly capable of being implanted in any vascular structure such as an aneurysm. The sensor assembly may include one or more sensors and/or antennas. The antenna may be in electrical communication with the sensor. The sensor may continuously or intermittently detect one or more physiological parameters of a patient and generate sensor data. The antenna may continuously or intermittently transmit sensor data related to the one or more physiological parameters of the patient to a receiver, which may be located within the patient's body or outside of the patient's body. The antenna may be capable of stabilizing a position of the sensor in the vascular structure.

The sensor assembly described above may include one or more following features. The antenna may be capable of being compressed (for instance, about the sensor) for loading into a delivery system and expanded when released from the delivery system. The antenna may at least partially or entirely surround the sensor. The antenna may extend across one or more surfaces of the sensor. For example, the antenna may form a single axis loop, a dual axis loop, or a spherical loop around the sensor. The antenna may transmit and/or receive RF signals. Signals received by the antenna may adjust operation of the sensor assembly, for example the frequency or type of physiological parameter(s) being monitored or power management of the sensor assembly. In some configurations, the sensor may only perform a function upon receipt of a command from outside the patient's body. The antenna may include platinum metal, platinum/iridium alloy, and/or nitinol. The antenna may be coated in a parylene film, a gold material, and/or a platinum material. In one embodiment the antenna is formed in part or wholly from platinum and iridium, such as a platinum/iridium alloy, e.g., an approximately 80/20 platinum/iridium alloy, e.g., about 75-85 parts platinum and about 15-25 parts iridium, or an approximately 90/10 platinum/iridium alloy, e.g., about 85-95 parts platinum and about 5-15 parts iridium. In one embodiment the antenna is formed from a gold outer cladding with nitinol inside.

The sensor may include a radiopaque marker to identify a location of the sensor in the patient. The sensor assembly may include a blood flow sensor, a blood pressure sensor, a metabolic sensor, a glucose sensor, an oxygen sensor, or other sensor. The sensor may generate the sensor data based on analyte materials, analyte elements, byproducts caused by certain cellular interactions or exchanges or blood interactions or exchanges in blood, and/or kinetic information. For example, the sensor may be capable of detecting oxygen, carbon dioxide, potassium, iron, and/or glucose in the blood of the patient.

The sensor assembly may include a sealing layer to hermetically seal the sensor assembly. The sensor assembly may include a dissolving membrane layer that dissolves when in contact with blood of the patient. The dissolving membrane layer may release clot enhancers when the dissolving membrane layer dissolves.

The sensor assembly may include a power source, such as a battery or supercapacitor, provided in the sensor assembly. Alternatively, the sensor may be powered by power source outside the patient's body. In some configurations, the sensor may be an inductive sensor. The power source may be rechargeable. The sensor assembly may include a memory device for storing sensor data or computer-executable instructions to be executed by a processor of the sensor assembly. The processor may be onboard the sensor or separate from the sensor.

The sensor assembly may be capable of being implanted in an aneurysm. The sensor assembly may detect one or more physiological parameters indicative of blood flow into or out of the aneurysm and/or level of clotting within the aneurysm.

The sensor may be capable of providing a first output indicative of a first level of blood flow and a second output indicative of a second level of blood flow. The first output may indicate a lack of clotting in the vascular structure. In response to the first output, a clinician may choose to deliver an occlusion device or deliver coagulant promoting drugs. The second output may indicate clotting in the vascular structure. In some configurations, the sensor may be a conductive switch.

Certain aspects of the disclosure are directed toward an implantable sensor system including a sensor assembly having any of the features described herein. The implantable sensor system may include an anchor structure for maintaining a position of the sensor in a vascular structure. The sensor assembly may be disposed within an interior space defined by the anchor structure. The anchor structure may be a separate component from the sensor. When implanted, the antenna of the sensor assembly may contact the anchor structure. This contact may enhance sensor data transmission. In other configurations, the sensor assembly may be directly or indirectly coupled to the anchor structure. For example, the antenna and/or the sensor may be directly or indirectly coupled to the anchor structure.

The anchor structure may be capable of occluding the vascular structure. The anchor structure may include one or more coils. The anchor structure may include a mesh or woven structure. The anchor structure may include a basket structure.

The sensor assembly and/or the anchor structure may be capable of eluting a drug to facilitate occlusion. The drug may be eluted upon implantation or at a predetermined amount of time after implantation. The drug may be eluted in response to the data collected from the sensor.

Any of the sensor assemblies or systems described herein may carry a drug (e.g., a coagulant) capable of treating a vascular structure in a patient. The drug may be coated on or stored in a cavity in the sensor assembly or the anchor structure. The sensor system may include a memory device for storing a computer-executable instruction. The sensor system may include a processor in communication with the memory device. The computer-executable instruction, when executed by the processor causes the processor to cause release the drug from the sensor assembly or the anchor structure. The computer-executable instruction may activate a switch to release the drug carried by the implantable sensor system. The processor may include a wireless receiver as a part of or separate from the antenna. The processor may execute the computer-executable instruction upon receipt of a wireless transmission from outside the body. The processor may execute the computer-executable instruction after a pre-determined time following implantation of the implantable sensor system.

Any of the sensor or sensor assemblies described herein may be capable of wirelessly communicating with an electronic device (e.g., base station or other computing device) using Bluetooth™, WiFi, ZigBee, cellular telephony, medical implant communication service (“MICS”), the medical device radio communications service (“MedRadio”), or other protocols. The electronic device may include a memory device and a processor. The memory device may be configured to store an application. The processor may execute the application to perform any of the functions described herein. For example, the processor may wirelessly communicate with a sensor assembly implanted in the vascular structure. The processor may determine a value of the one or more physiological parameters indicative of blood flow. The processor may output for presentation on a display the value for presentation to a user. The value may provide a metric indicative of a degree to which the vascular structure is occluding. The processor may execute the application to communicate the value via a communication network to a computing system. The processor may execute the application to transmit a setting adjustment command to the sensor assembly to adjust a setting for monitoring the one or more physiological parameters, for example the timing of collecting data. Additionally or alternatively, the setting adjustment command may adjust a different operation of the sensor assembly, such as power management. In some configurations, the sensor assembly may only collect data in response to a command received from the processor.

Certain aspects of the disclosure are directed toward a kit including an implantable sensor assembly and/or anchor structure having any of the features described herein. The kit may also include a delivery system capable of releasing the implantable sensor assembly and/or the anchor structure in the vascular structure. The same or different delivery systems may be used to deliver the sensor assembly and the anchor structure. Optionally, the kit may include the electronic device described herein.

Certain aspects of the disclosure are directed toward a method of monitoring a vascular structure using any of the sensor assemblies described herein. The vascular structure may include a neurovascular or cardiovascular structure, including but not limited to an aneurysm of an artery in a posterior circulation of the patient, a basilar aneurysm, a bifurcation aneurysm, a sidewall aneurysm, a ductus arteriosus, a carotid artery, or a venous structure. The method may include continuously or intermittently detecting one or more physiological parameters in the vascular structure using the implantable sensor assembly. The method may include continuously or intermittently transmitting sensor data related to the one or more physiological parameters to a remote location within the patient's body or outside the patient's body.

The method described above may include one or more following steps. The method may include occluding the vascular structure with an anchor structure. The sensor assembly may include a blood flow sensor, a blood pressure sensor, a metabolic sensor, a glucose sensor, an oxygen sensor, or other sensor. The method may include generating sensor data based on analyte materials, analyte elements, byproducts caused by certain cellular interactions or exchanges or blood interactions or exchanges in blood, and/or kinetic information. The sensor assembly may detect one or more physiological parameters, including but not limited to, oxygen, carbon dioxide, potassium, iron, and/or glucose in the blood of the patient and transmit the one or more physiological parameters using one or more antennas. The method may include recharging the sensor assembly.

Certain aspects of this disclosure are directed toward a method of implanting any of the sensor assemblies into a vascular structure of a patient. The method may include percutaneously advancing a delivery system carrying a sensor system to a vascular structure and releasing the sensor assembly into the vascular structure. The method may include advancing the delivery system over a guidewire or through a guide catheter. The delivery system may include a pusher to push the sensor assembly out of the delivery system. The method may also include simultaneously or separately releasing an anchor structure into the vascular structure. Releasing the anchor structure may include positioning the anchor structure around the sensor assembly. In other methods, the sensor assembly may be released in an interior space within the anchor structure. The anchor structure may occlude the vascular structure (such as, limit or prevent blood flow).

Certain aspects of this disclosure are directed toward a delivery system for delivering any of the sensor assemblies and/or systems described herein to a vascular structure. The delivery system can include a handle, a distal tip, and a shaft therebetween. The distal tip may be actively or passively deflected. In embodiments with active deflection, the distal tip may steered using mechanical and/or electronic controls. The distal tip may include a loading chamber for carrying the sensor assembly. The handle may include one or more user-actuatable mechanisms to release the sensor assembly and/or sensor system, control the distal tip, and/or stabilize the delivery system. The shaft may include one or more lumens, for example a guidewire lumen, a fluid delivery lumen, steering lumen, and/or a lumen for scarrying a pusher. In some configurations, the delivery system includes a delivery sleeve positioned over the shaft. The delivery sleeve may directly or indirectly cause deflection of the distal tip.

Certain aspects of this disclosure are directed toward an implantable system for treating a cranial aneurysm. The implantable system can include a plurality of coils configured to be positioned within the cranial aneurysm to cause an occlusion of a flow of blood in the cranial aneurysm. The implantable system can include a sensor configured to be positioned within the cranial aneurysm and further configured to detect a property of the flow of blood. The implantable system can include a communication circuitry configured to be positioned within the cranial aneurysm, the communication circuitry connected to the sensor and configured to transmit data related to the property of the flow of blood detected by the sensor. The implantable system can include an antenna configured to be positioned within the cranial aneurysm, the antenna connected to the communication circuitry and further configured to facilitate transmission of the data related to the property of the flow of blood detected by the sensor.

Certain aspects of this disclosure are directed toward an implantable system for monitoring a vascular structure. The implantable system can include a sensor configured to be positioned within the vascular structure and further configured to detect a property of a flow of blood in the vascular structure. The implantable system can include a communication circuitry configured to be positioned within the vascular structure, the communication circuitry connected to the sensor and configured to transmit data related to the property of the flow of blood detected by the sensor. The implantable system can include an antenna configured to be positioned within the vascular structure, the antenna connected to the communication circuitry and further configured to facilitate transmission of the data related to the property of the flow of blood detected by the sensor.

### BRIEF DESCRIPTION OF THE DRAWINGS

Example features of the present disclosure, its nature and various advantages will be apparent from the accompanying drawings and the following detailed description of various embodiments. Non-limiting and non-exhaustive embodiments are described with reference to the accompanying drawings, wherein like labels or reference numbers refer to like parts throughout the various views unless otherwise specified. The sizes and relative positions of elements in the drawings are not necessarily drawn to scale. For example, the shapes of various elements are selected, enlarged, and positioned to improve drawing legibility. The particular shapes of the elements as drawn have been selected for ease of recognition in the drawings. One or more embodiments are described hereinafter with reference to the accompanying drawings in which:

FIG. 1 illustrates an example sensor environment, according to certain aspects of the present disclosure.

FIGS. 2A, 2B, 2C and 2D illustrate example sensor systems implanted in an aneurysm, according to certain aspects of the present disclosure.

FIG. 3 illustrates a top view of a sensor assembly, according to certain aspects of the present disclosure.

FIG. 4 illustrates an antenna modeling simplification, according to certain aspects of the present disclosure.

FIGS. 5A, 5B, 5C and 5D illustrate modeling a meander monopole antenna, according to certain aspects of the present disclosure.

FIGS. 6A, 6B, 6C, 6D, 6E, 6F, 6G and 6H illustrate modeling a flat monopole antenna, according to certain aspects of the present disclosure.

FIGS. 7A, 7B, 7C, 7D, 7E, 7F, 7G, 7H, 7I and 7J illustrate modeling a planar inverted F-antenna, according to certain aspects of the present disclosure.

FIG. 8 is a table summarizing performance of various antenna configurations, according to certain aspects of the present disclosure.

FIGS. 9A, 9B, and 9C illustrate planar inverted F-antennas, according to certain aspects of the present disclosure.

FIGS. 10A, 10B, 10C, 10D, 10E, 10F, 10G and 10H illustrate single-antenna and multiple-antenna configurations, according to certain aspects of the present disclosure.

FIGS. 11 and 12 illustrates flowcharts of methods of implanting an example sensor system into a patient, according to certain aspects of the present disclosure.

FIG. 13 illustrates a delivery system according to certain aspects of the present disclosure.

FIG. 14 illustrates another delivery system according to certain aspects of the present disclosure.

### DETAILED DESCRIPTION

The present disclosure may be understood more readily by reference to the following detailed description of example configurations of “a sensor assembly” or “a sensor system” included herein. The following description, along with the accompanying drawings, sets forth certain specific details in order to provide a thorough understanding of various disclosed embodiments. However, one skilled in the relevant art will recognize that the disclosed embodiments may be practiced in various combinations, without one or more of these specific details, or with other methods, components, devices, materials, etc. In other instances, well-known structures or components that are associated with the environment of the present disclosure, including but not limited to the communication systems and networks, have not been shown or described in order to avoid unnecessarily obscuring descriptions of the embodiments. Additionally, the various embodiments may be methods, systems, media, or devices. Accordingly, the various embodiments may be entirely hardware embodiments, entirely software embodiments, entirely firmware embodiments, or embodiments combining or subcombining software, firmware, and hardware aspects.

Prior to setting forth this disclosure in more detail, it may be helpful to an understanding thereof to provide definitions of certain terms to be used herein. Additional definitions are set forth throughout this disclosure. The terms “include” and “comprise,” as well as derivatives thereof, mean inclusion without limitation. The term “or,” is inclusive, meaning and/or. The phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like. The term “controller” or “processor” means any device, system, or part thereof that controls at least one operation, such a device may be implemented in hardware (e.g., electronic circuitry), firmware, or software, or some combination of at least two of the same. The functionality associated with any particular controller may be centralized or distributed, whether locally or remotely. Other definitions of certain words and phrases may be provided within this patent document. Those of ordinary skill in the art will understand that in many, if not most instances, such definitions apply to prior as well as future uses of such defined words and phrases.

An “intelligent medical device” as used in the present disclosure, is an implantable or implanted medical device that desirably replaces or functionally supplements a subject's natural body part. The intelligent medical device can include one of the disclosed sensor assemblies and/or an anchoring (or anchor) structure (e.g., a metal coil or basket). The sensor assembly will comprise or be associated with a controller or processor, also referred to as an implantable reporting processor (“IRP”). In one configuration, the intelligent medical device is an implanted or implantable medical device having a sensor assembly with the IRP arranged to perform the functions as described herein. The sensor assembly may perform one or more of the following example actions in order to characterize the post-implantation status of the intelligent medical device: identifying the intelligent medical device or a portion of the intelligent medical device (e.g., the sensor assembly or by recognizing one or more unique identification codes for the intelligent medical device or a portion of the intelligent medical device); detecting, sensing and/or measuring parameters, which may collectively be referred to as monitoring parameters, in order to collect operational, physiological, kinematic or other data about the intelligent medical device or a portion of the intelligent prosthesis (e.g., the sensor assembly) and such data may optionally be collected as a function of time; storing the collected data within the intelligent medical device or a portion of the intelligent medical device (e.g., the sensor assembly); and communicating the collected data and/or the stored data by a wireless means from the intelligent medical device or a portion of the intelligent medical device (e.g., the sensor assembly) to an external computing device. The external computing device may have or otherwise have access to at least one data storage location such as found on a personal computer, a base station, a computer network, a cloud-based storage system, or another computing device that has access to such storage. Non-limiting and non-exhaustive list of configurations of intelligent medical devices include a metal coil configured to be implanted in an aneurysm.

“Monitoring data,” as used herein, individually or collectively includes some or all data associated with a particular implantable sensor assembly, and available for communication outside of the particular implantable sensor system. For example, monitoring data may include raw data from one or more sensors of the sensor assembly. The one or more sensors can be configured to detect analyte materials in the blood (e.g., glucose), analyte elements in the blood (e.g., oxygen or carbon dioxide), and the like that produce data associated with one or more physiological parameters of a patient. For example, the one or more physiological parameters of the patient can be associated with the patient's aneurysm, after it has been treated via coil embolization or the like. Monitoring data may also include processed data from one or more sensors, status data, operational data, control data, fault data, time data, scheduled data, event data, log data, and the like associated with the particular sensor assembly. In some cases, high resolution monitoring data includes monitoring data from one, many, or all of the sensors of the sensor assembly that is collected in higher quantities, resolution, from more sensors, more frequently, or the like.

“Sensor” refers to a device that can be utilized to do one or more of detect, measure and/or monitor one or more different aspects of a body tissue (e.g., anatomy, physiology, metabolism, and/or function) and/or one or more aspects of the smart medical device or the sensor system. Representative examples of sensors suitable for use within the present invention include, for example, oxygen sensors, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), accelerometers, mechanical stress sensors and temperature sensors. Within certain configurations the sensor can be a wireless sensor, or, within other configurations, a sensor connected to a wireless microprocessor. Within further configuration one or more (including all) of the sensors can have a Unique Sensor Identification number (“USI”) which specifically identifies the sensor.

“Sensor assembly” may refer to one or more components. For example, the “sensor assembly” may be a single component sensor with processing and wireless transmission on board the sensor. In other examples, the “sensor assembly” may be multiple components with a sensor and other components for performing one or more functions described herein.

In order to further understand the various aspects of the invention provided herein, the following sections are provided below: I. Overview; II. Sensor Assembly; A. Antenna(s); B. Sensor; C. Processor/Controller; D. Power Source; III. Methods of Use of the Sensor System; IV. Kit; V. Delivery System; VI. Additional Embodiments and Terminology; and VII. Example Embodiments.

### 1. OVERVIEW

The apparatuses, systems, and methods disclosed in the present disclosure can be used in treating a vascular structure in a patient's vascular system, such as in brain, lungs, chest, or thoracic region, among others. Example vascular structures include neurovascular, respiratory, or cardiovascular structures, including but not limited to: an aneurysm, a carotid artery, a venous structure, a ductus arteriosus, or other vascular structure. Example aneurysms include: an aneurysm of an artery in a posterior circulation of the patient, a basilar aneurysm, a bifurcation aneurysm, an intracranial aneurysm, and a sidewall aneurysm. It can be difficult to monitor a treatment of an aneurysm, especially an intracranial aneurysm. For instance, multiple MRI tests (or other tests) may need to be performed subsequent to preforming the surgical procedure for treating the aneurysm. This can be costly, time consuming, and burdensome. Thus, it would be useful to implant an intelligent medical device and/or a sensor system with the implant (e.g., metal coil) in the aneurysm to monitor treatment. Parameters monitored by the sensor system can be wirelessly transmitted using one or more antennas of the sensor system, thereby allowing monitoring without the need to perform MRI tests (or other tests).

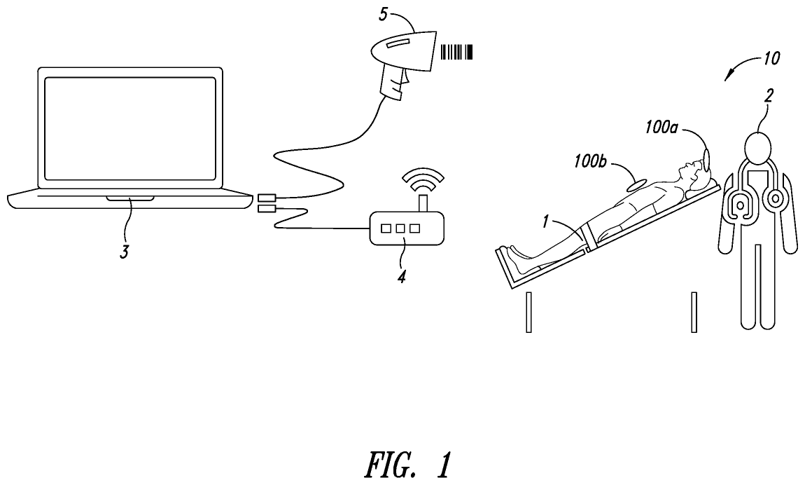

FIG. 1 illustrates an example sensor environment 10. In the environment 10, one or more sensor assemblies 100a, 100b can be implanted by a medical practitioner 2 in the body of a patient 1. The sensor assembly 100a, 100b can include an associated implantable reporting processor (“IPR”) that can be arranged and configured to collect data including for example, medical and health data related to the patient 1 which the sensor assembly 100a, 100b is associated, and operational data of the sensor assembly 100a, 100b itself. The sensor assembly 100a, 100b can communicate with one or more base stations 4 or one or more computing devices 3 during different stages of monitoring the patient 1. While implanted in the patient's body 1, the sensor assembly 100a, 100b can also communicate with a barcode scanner 5 such that the barcode scanner 5 can identify the particular assembly 100a, 100b implanted in the patient 1. The barcode scanner 5 and/or the base station 4 can communicate with the one or more computing devices 3.

For example, in association with a medical procedure, the sensor assembly 100a, 100b can be implanted in the patient's body 1. The sensor assembly 100a, 100b can communicate with an operating room base station 4. While the patient 1 is at home and after sufficient recovery from the medical procedure, the sensor assembly 100a, 100b can be arranged to communicate with a home base station (not shown) and/or a doctor office base station (not shown). The sensor assembly 100a, 100b can communicate with each base station via a short range network protocol, such as the medical implant communication service (“MICS”), the medical device radio communications service (“MedRadio”), Bluetooth, or some other wireless communication protocol suitable for use with the sensor assembly 100a, 100b.

The sensor assembly 100a, 100b may be a standalone medical device or it may be a component in a larger system, including an anchor structure such as a metal coil or basket that can desirably collect and provide in situ-patient medical data, device operational data, or other useful data.

The sensor assembly 100a, 100b can include one or more measurement units, for example a sensor, that can collect information and data, including medical and health data related to a patient 1 which the sensor assembly 100a, 100b is associated, and operational data of the assembly 100a, 100b itself.

The sensor assembly 100a, 100b can collect data at various different times and at various different rates during a monitoring process of the patient 1. In some configurations, the sensor assembly 100a, 100b may operate in a plurality of different phases over the course of monitoring the patient. For example, the sensor assembly 100a, 100b can collect more data soon after the sensor assembly 100a, 100b is implanted into the patient 1 and less data as the patient 1 heals and thereafter.

The amount and type of data collected by a sensor assembly 100a, 100b may be different from patient to patient, and the amount and type of data collected may change for a single patient. For example, a medical practitioner studying data collected by the sensor assembly 100a, 100b of a particular patient may adjust or otherwise control how the sensor assembly 100a, 100b collects future data.

The amount and type of data collected by a sensor assembly 100a, 100b may be different for different body parts, for different types of patient conditions, for different patient demographics, or for other differences. Alternatively, or in addition, the amount and type of data collected may change overtime based on other factors, such as how the patient is healing or feeling, how long the monitoring process is projected to last, how much battery power remains and should be conserved, the type of movement being monitored, the body part being monitored, and the like. In some cases, the collected data can be supplemented with personally descriptive information provided by the patient such as subjective pain data, quality of life metric data, co-morbidities, perceptions or expectations that the patient associates with the sensor assembly 100a, 100b, or the like.

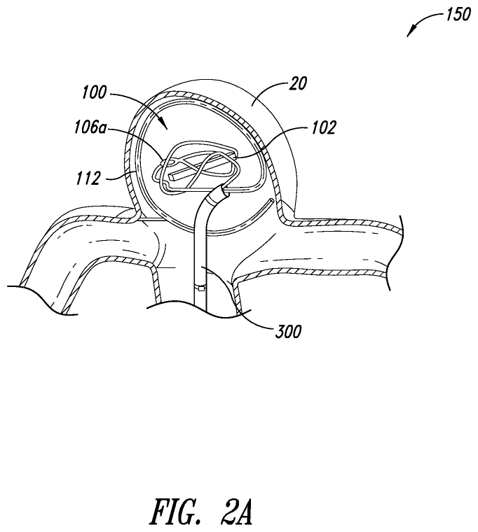

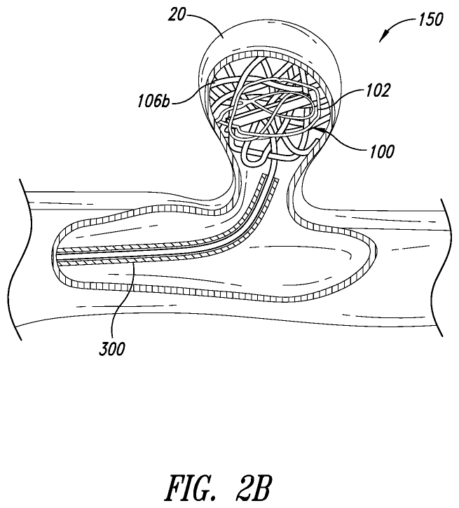

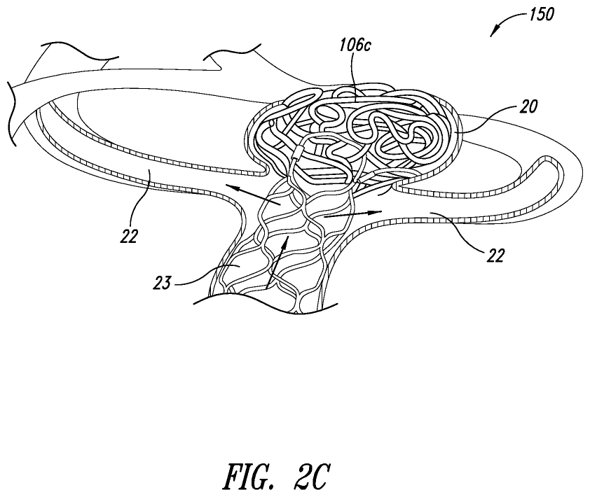

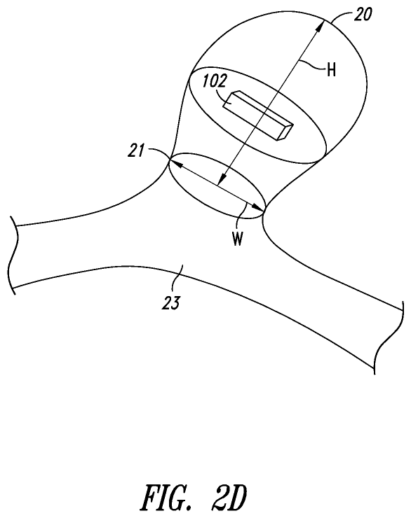

Implantation of the sensor assembly 100a, 100b into the patient 1 may occur in an operating room, as shown in FIG. 2. As used herein, operating room includes any office, room, building, or facility where the sensor assembly 100a, 100b can be implanted into the patient. For example, the operating room may be a typical operating room in a hospital, an operating room in a surgical clinic or a doctor's office, or any other operating theater, interventional suite, intensive care ward, emergency room or the like where the sensor assembly 100a, 100b is implanted into the patient.

The operating room base station 4 can be utilized to configure and initialize the sensor assembly 100a, 100b when the sensor assembly 100a, 100b is being implanted into the patient 1. A communicative relationship can be formed between the sensor assembly 100a, 100b and the operating room base station 4, for example, based on a polling signal transmitted by the operating room base station 4 and a response signal transmitted by the sensor assembly 100a, 100b.

Upon forming a communicative relationship, which can often occur prior to implantation of the sensor assembly 100a, 100b, the operating room base station 4 can transmit initial configuration information to the sensor assembly 100a, 100b. The initial configuration information may include, but is not limited to, a time stamp, a day stamp, an identification of the type and placement of the sensor assembly 100a, 100b, information on other implants associated with the sensor assembly 100a, 100b, surgeon information, patient identification, operating room information, and the like.

In some configurations, the initial configuration information can be passed unidirectionally. In some embodiments, the initial configuration information can be passed bidirectionally. The initial configuration information may define at least one parameter associated with the collection of data by the sensor assembly 100a, 100b. For example, the initial configuration information may identify settings for one or more sensors of the sensor assembly 100a, 100b for each of one or more modes of operation. The initial configuration information may also include other control information, such as an initial mode of operation of the sensor assembly 100a, 100b, a particular event that triggers a change in the mode of operation, radio settings, data collection information (e.g., how often the sensor assembly 100a, 100b wakes up to collected data, how long it collects data, how much data to collect), home base station (not shown), computing device 3, and a connected personal assistant identification information, and other control information associated with the implantation or operation of the sensor assembly 100a, 100b. Examples of a connected personal assistant, which also can be called a smart speaker, include Amazon Echo®, Amazon Dot®, Google Home®, Philips® patient monitor, Comcast's health-tracking speaker, and Apple HomePod®.

In some configurations, the initial configuration information may be pre-stored on the operating room base station 4 or an associated computing device 3. In other configurations, a surgeon, surgical technician, or some other medical practitioner 2 may input the control information and other parameters to the operating room base station 4 for transmission to the sensor assembly 100a, 100b. In at least one such configuration, the operating room base station 4 may communicate with an operating room configuration computing device 3. The operating room configuration computing device 3 can include an application with a graphical user interface that enables the medical practitioner to input configuration information for the sensor assembly 100a, 100b. In various configurations, the application executing on the operating room configuration computing device 3 may have some of the configuration information predefined, which may or may not be adjustable by the medical practitioner 2. The operating room configuration computing device 3 can communicate the configuration information to the operating room base station 4 via a wired, as shown in FIG. 1, or wireless network connection (e.g., via a USB connection, Bluetooth connection, Bluetooth Low Energy (“BTLE”) connection, or Wi-Fi connection), which can communicate it to the sensor assembly 100a, 100b.

The operating room configuration computing device 3 may also display information regarding the sensor assembly 100a, 100b or the operating room base station 4 to the surgeon, surgical technician, or other medical practitioner 2. For example, the operating room configuration computing device 3 may display error information if the sensor assembly 100a, 100b is unable to store or access the configuration information, if the sensor assembly 100a, 100b is unresponsive, if the sensor assembly 100a, 100b identifies an issue with one of the sensors or radio during an initial self-test, if the operating room base station 4 is unresponsive or malfunctions, or for other reasons.

Although the operating room base station 4 and the operating room configuration computing device 3 are described as separate devices, embodiments are not so limited; rather, the functionality of the operating room configuration computing device 3 and the operating room base station 4 may be included in a single computing device or in separate devices as illustrated. In this way, the medical practitioner 1 may be enabled in one embodiment to input the configuration information directly into the operating room base station 4.

Returning to FIG. 1, once the sensor assembly 100a, 100b is implanted into the patient and the patient returns home, the home base station, the computing or smart device (e.g., the patient's smart phone), the connected personal assistant, or two or more of the home base station, and the computing or smart device, and the connected personal assistant can communicate with the sensor assembly 100a, 100b. The sensor assembly 100a, 100b can collect data at determined rates and times, variable rates and times, or otherwise controllable rates and times. Data collection can start when the sensor assembly 100a, 100b is initialized in the operating room, when directed by a medical practitioner 1, or at some later point in time. At least some data collected by the sensor assembly 100a, 100b may be transmitted to the home base station directly, to the smart device directly, to the connected personal assistant directly, to the base station via one or both of the smart device and the connected personal assistant, to the smart device via one or both of the base station and the connected personal assistant, or to the connected personal assistant via one or both of the smart device and the base station. Here, “one or both” means via an item alone, and via both items serially or in parallel. For example, data collected by the sensor assembly 100a, 100b may be transmitted to the home base station via the smart device alone, via the connected personal assistant alone, serially via the smart device and the connected personal assistant, serially via the connected personal assistant and the smart device, and directly, and possibly contemporaneously, via both the smart device and the connected personal assistant. Similarly, data collected by the sensor assembly 100a, 100b may be transmitted to the smart device via the home base station alone, via the connected personal assistant alone, serially via the home base station and the connected personal assistant, serially via the connected personal assistant and the home base station, and directly, and possibly contemporaneously, via both the home base station and the connected personal assistant. Further in example, data collected by the sensor assembly 100a, 100b may be transmitted to the connected personal assistant via the smart device alone, via the home base station alone, serially via the smart device and the home base station, serially via the home base station and the smart device, and directly, and possibly contemporaneously, via both the smart device and the home base station.

In various configurations, one or more of the home base station, the smart device, and the connected personal assistant can ping the sensor assembly 100a, 100b at periodic, predetermined, or other times to determine if the sensor assembly 100a, 100b is within communication range of one or more of the home base station, the smart device, and the connected personal assistant. Based on a response from the sensor assembly 100a, 100b, one or more of the home base station, the smart device, and the connected personal assistant determines that the sensor assembly 100a, 100b is within communication range, and the sensor assembly 100a, 100b can be requested, commanded, or otherwise directed to transmit the data it has collected to one or more of the home base station, the smart device, and the connected personal assistant.

Each of one or more of the home base station, the smart device, and the connected personal assistant may, in some cases, be arranged with a respective optional user interface. The user interface may be formed as a multimedia interface that unidirectionally or bi-directionally passes one or more types of multimedia information (e.g., video, audio, tactile, etc.). Via the respective user interface of one or more of the home base station, the smart device, and the connected personal assistant, the patient 1 or an associate (not shown in FIG. 1) of the patient 1 may enter other data to supplement the data collected by the sensor assembly 100a, 100b. A user, for example, may enter personally descriptive information (e.g., age change, weight change), changes in medical condition, co-morbidities, pain levels, quality of life, or other subjective metric data, personal messages for a medical practitioner, and the like. In these configurations, the personally descriptive information may be entered with a keyboard, mouse, touch-screen, microphone, wired or wireless computing interface, or some other input means. In cases where the personally descriptive information is collected, the personally descriptive information may include, or otherwise be associated with, one or more identifiers that associate the information with unique identifier of the sensor assembly 100a, 100b, the patient, an associated medical practitioner, an associated medical facility, or the like.

In some of these cases, a respective optional user interface of each of one or more of the home base station, the smart device, and the connected personal device may also be arranged to deliver information associated with the sensor assembly 100a, 100b to the user from, for example, a medical practitioner 2. In these cases, the information delivered to the user may be delivered via a video screen, an audio output device, a tactile transducer, a wired or wireless computing interface, or some other like means.

In configurations where one or more of the home base station, the smart device, and the connected personal assistant are arranged with a user interface, which may be formed with an internal user interface arranged for communicative coupling to a patient portal device. The patent portal device may be smartphone, a tablet, a body-worn device, a weight or other health measurement device (e.g., thermometer, bathroom scale, etc.), or some other computing device capable of wired or wireless communication. In these cases, the user is able to enter the personally descriptive information, and the user also may be able to receive information associated with the sensor assembly 100a, 100b.

The home base station can utilize a home network of the patient to transmit the collected data to cloud. The home network, which may be a local area network, provides access from the home of the patient to a wide area network, such as the internet. In some configurations, the home base station may utilize a Wi-Fi connection to connect to the home network and access the internet. In other embodiments, the home base station may be connected to a home computer (not shown) of the patient, such as via a USB connection, which itself is connected to the home network.

The smart device can communicate with the sensor assembly 100a, 100b directly via, for example, Bluetooth® compatible signals, and can utilize the home network of the patient to transmit the collected data to cloud, or can communicate directly with the cloud, for example, via a cellular network. Alternatively, the smart device can be configured to communicate directly with one or both of the base station and the connected personal assistant via, for example, Bluetooth® compatible signals, and is not configured to communicate directly with the sensor assembly 100a, 100b.

Furthermore, the connected personal assistant can communicate with the sensor assembly 100a, 100b directly via, for example, Bluetooth® compatible signals, and can utilize the home network of the patient to transmit the collected data to cloud, or can communicate directly with the cloud, for example, via a modem/internet connection or a cellular network. Alternatively, the connected personal assistant can be configured to communicate directly with one or both of the base station and the smart device via, for example, Bluetooth® compatible signals, and not configured to communicate directly with the sensor assembly 100a, 100b.

Along with transmitting collected data to the cloud, one or more of the home base station, the smart device, and the connected personal assistant may also obtain data, commands, or other information from the cloud directly or via the home network. One or more of the home base station, the smart device, and the connected personal assistant may provide some or all of the received data, commands, or other information to the sensor assembly 100a, 100b. Examples of such information include, but are not limited to, updated configuration information, diagnostic requests to determine if the sensor assembly 100a, 100b is functioning properly, data collection requests, and other information.

The cloud may include one or more server computers or databases to aggregate data collected from the sensor assembly 100a, 100b, and in some cases personally descriptive information collected from a patient, with data collected from other intelligent implantable devices, and in some cases personally descriptive information collected from other patients. In this way, the cloud can create a variety of different metrics regarding collected data from each of a plurality of intelligent implantable devices that are implanted into separate patients. This information can be helpful in determining if the intelligent implantable devices are functioning properly. The collected information may also be helpful for other purposes, such as determining which specific devices may not be functioning properly, determining if a procedure or condition associated with the intelligent implantable device is helping the patient (e.g., if a sensor system, which includes a sensor assembly 100a, 100b, is operating properly), and determining other medical information.

At various times throughout the monitoring process, the patient may be requested to visit a medical practitioner for follow up appointments. This medical practitioner may be the surgeon who implanted the sensor assembly 100a, 100b in the patient or a different medical practitioner that supervises the monitoring process, physical therapy, and recovery of the patient. For a variety of different reasons, the medical practitioner may want to collect real-time data from the sensor assembly 100a, 100b in a controlled environment. In some cases, the request to visit the medical practitioner may be delivered through a respective optional bidirectional user interface of each of one or more of the home base station, the smart device, and the connected personal assistant.

A medical practitioner can utilize the doctor office base station, which communicates with the sensor assembly 100a, 100b, to pass additional data between the doctor office base station and the sensor assembly 100a, 100b. Alternatively, or in addition, the medical practitioner can utilize the doctor office base station to pass commands to the sensor assembly 100a, 100b. In some configurations, the doctor office base station can instruct the sensor assembly 100a, 100b to enter a high-resolution mode to temporarily increase the rate or type of data that is collected for a short time. The high-resolution mode directs the sensor assembly 100a, 100b to collect different (e.g., large) amounts of data during an activity where the medical practitioner is also monitoring the patient.

In some configurations, the doctor office base station can enable the medical practitioner to input event markers, which can be synchronized with the high-resolution data collected by the sensor assembly 100a, 100b. For example, assume the sensor assembly 100a, 100b is a component in a sensor system adapted to be implanted into an intracranial aneurysm. During a follow up visit, the medical practitioner can put the sensor assembly 100a, 100b in the high-resolution mode. The medical practitioner can review the sensor data from the sensor assembly 100a, 100b and determine whether the aneurysm is clotting. If the sensor data indicates that the aneurysm is not clotting the medical practitioner can administer medication to the patient. The medical practitioner could administer beta blockers and/or calcium channel blockers to lower the patient's blood pressure and relax their blood vessels. Alternatively, the medical practitioner may administer antifibrinolytic drugs (e.g., aprotinin, tranexamic acid, epsilon-aminocaproic acid) that promote blood clotting. After the medical practitioner administers the medication to the patient, the medical practitioner can click an event marker button on the doctor office base station to mark the administration of the medication. The doctor office base station records the marker and the time at which the marker was input. When the timing of this marker is synchronized with the timing of the collected high-resolution data, the medical practitioner can analyze the data to try and determine the effects of the medication.

In other configurations, the doctor office base station may provide updated configuration information to the sensor assembly 100a, 100b. The sensor assembly 100a, 100b can store this updated configuration information, which can be used to adjust the parameters associated with the collection of the data. For example, if the patient is doing well, the medical practitioner can direct a reduction in the frequency at which the sensor assembly 100a, 100b collects data. On the contrary, if the defect or injury is not healing (e.g., the aneurysm is not clotting), the medical practitioner may direct the sensor assembly 100a, 100b to collect additional data for a determined period of time (e.g., a few days). The medical practitioner may use the additional data to diagnose and treat a particular problem. In some cases, the additional data may include personally descriptive information provided by the patient after the patient has left presence of the medical practitioner and is no longer in range of the doctor office base station. In these cases, the personally descriptive information may be collected and delivered from via one or more of the home base station, the smart device, and the connected personal assistant. Firmware within the sensor assembly 100a, 100b and/or the base station can provide safeguards limiting the duration of such enhanced monitoring to insure the battery retains sufficient power to last for the implant's lifecycle. Additionally, or alternatively, the sensor assembly 100a, 100b can include a conductive switch, which is further described below, that assists in limiting the monitoring of the sensor assembly 100a, 100b.

In various configurations, the doctor office base station may communicate with a doctor office configuration computing device. The doctor office configuration computing device can include an application with a graphical user interface that enables the medical practitioner to input commands and data. Some or all of the commands, data, and other information may be later transmitted to the sensor assembly 100a, 100b via the doctor office base station. For example, in some configurations, the medical practitioner can use the graphical user interface to instruct the sensor assembly 100a, 100b to enter its high-resolution mode. In other configurations, the medical practitioner can use graphical user interface to input or modify the configuration information for the sensor assembly 100a, 100b. The doctor office configuration computing device can transmit the information (e.g., commands, data, or other information) to the doctor office base station via a wired or wireless network connection (e.g., via a USB connection, Bluetooth® connection, or Wi-Fi connection), which in turn can transmits some or all of the information to the sensor assembly 100a, 100b.

The doctor office configuration computing device may also display, to the medical practitioner, other information regarding the sensor assembly 100a, 100b, regarding the patient (e.g., personally descriptive information), or the doctor office base station. For example, the doctor office configuration computing device may display the high-resolution data that is collected by the sensor assembly 100a, 100b and transmitted to the doctor office base station. The doctor office configuration computing device may also display error information if the sensor assembly 100a, 100b is unable to store or access the configuration information, if the sensor assembly 100a, 100b is unresponsive, if the sensor assembly 100a, 100b identifies an issue with one of the sensors or radio, if the doctor office base station is unresponsive or malfunctions, or for other reasons.

In some configurations, doctor office configuration computing device may have access to the cloud. In at least one embodiment, the medical practitioner can utilize the doctor office configuration computing device to access data stored in the cloud, which was previously collected by the sensor assembly 100a, 100b and transmitted to the cloud via one or both of the home base station and the smart device. Similarly, the doctor office configuration computing device can transmit the high-resolution data obtain from the sensor assembly 100a, 100b via the doctor office base station to the cloud. In some configurations, the doctor office base station may have internet access and may be enabled to transmit the high-resolution data directly to the cloud without the use of the doctor office configuration computing device.

In various configurations, the medical practitioner may update the configuration information of the sensor assembly 100a, 100b when the patient is not in the medical practitioner's office. In these cases, the medical practitioner can utilize the doctor office configuration computing device to transmit updated configuration information to the sensor assembly 100a, 100b via the cloud. One or more of the home base station, the smart device, and the connected personal assistant can obtain updated configuration information from the cloud and pass updated configuration information to the cloud. This can allow the medical practitioner to remotely adjust the operation of the sensor assembly 100a, 100b without needing the patient to come to the medical practitioner's office. This may also permit the medical practitioner to send messages to the patient in response, for example, to personally descriptive information that was provided by the patient and passed through one or more of the home base station, the smart device, and the connected personal assistant to the doctor office base station.

Although the doctor office base station and the doctor office configuration computing device are described as separate devices, configurations are not so limited; rather, the functionality of the doctor office configuration computing device and the doctor office base station may be included in a single computing device or in separate devices (as illustrated). In this way, the medical practitioner may be enabled in one configuration to input the configuration information or markers directly into the doctor office base station and view the high-resolution data (and synchronized marker information) from a display on the doctor office base station.

Still referring to FIG. 1, alternate configurations are contemplated. For example, each of the base station, the smart device, and the connected personal assistant may be configured to communicate with one or both of the sensor assembly 100a, 100b and the cloud via another one or two of the base station, the smart device, and the connected personal assistant. Moreover, the smart device can be temporarily contracted as an interface to the sensor assembly 100a, 100b, and can be any suitable device other than a smart phone, such as a smart watch, a smart patch, and any IoT device, such as a coffee pot, capable of acting as an interface to the sensor assembly 100a, 100b. In addition, one or more of the base station, smart device, and connected personal assistant can act as a communication hub for multiple sensor assemblies 100a, 100b implanted in one or more patients. Furthermore, one or more of the base station, smart device, and connected personal assistant can automatically order or reorder prescriptions or medical supplies (e.g., a calcium channel blocker) in response to patient input or sensor assembly 100a, 100b input (e.g., pain level, level of clotting) if a medical professional and insurance company have preauthorized such an order or reorder; alternatively, one or more of the base station, smart device, and connected personal assistant can be configured to request, from a medical professional or an insurance company, authorization to place the order or reorder. Moreover, one or more of the base station, smart device, and connected personal assistant can be configured with a personal assistant such as Alexa® or Siri®.

### II. SENSOR ASSEMBLY

Any of the sensor assemblies described herein are capable of being implanted within any vascular structure, such as an aneurysm.

As shown in FIGS. 2A-2C, a sensor assembly 100 can include one or more sensors 102 and/or one or more antennas 112 (sometimes referred to in this disclosure in a singular form as an antenna 112). The one or more sensors 102 can monitor one or more physiological parameters. The one or more physiological parameters may be indicative of blood flow through a vascular system of a patient or other conditions of the patient. For example, the one or more sensor 102 can monitor the one or more physiological parameters continuously, intermittently at a regular time interval, or responsive to receiving a command. The one or more antennas 112 can transmit the sensor data to a receiver outside the patient's body. For example, the one or more antennas 112 can transmit sensor data continuously, intermittently at a regular time interval, or responsive to receiving a command.

The sensor assembly 100 can monitor one or more physiological parameters indicative of blood flow through a vascular structure, such as an aneurysm 20 as shown in FIGS. 2A-2C. The sensor assembly 100 may include one or more sensors to monitor other conditions of the patient, such as movement. For example, the sensor assembly 100 may include an accelerometer to monitor whether the patient is standing, sitting, or laying down.

The sensor assembly 100 may be included in a sensor system 150, which can also include an anchor (or anchoring) structure. For example, FIG. 2A illustrates a sensor system 150 being implanted into the aneurysm 20 by a delivery system 300 (such as, a catheter). Anchor structure 106A may be positioned within the aneurysm 20, such as between the sensor assembly 100 and a wall of the aneurysm 20. The anchor structure 106A may take the form of one or more framing coils or other structures to maintain an entrance of the aneurysm 20 and/or stabilize the aneurysm. For example, the anchor structure 106A may at least partially surround sensor assembly 100 and contact the wall of the aneurysm 20. Viewed another way, the sensor assembly 100 may be disposed within an interior space defined by the anchor structure 106A. The anchor structure 106A may directly or indirectly contact the sensor assembly 100. For example, the anchor structure 106A may contact the antenna 112 and/or the sensor 102. In some configurations, the anchor structure 106A may be directly or indirectly coupled to the sensor assembly 100, such as to the antenna 112. In other configurations, the anchor structure 106A may not contact the sensor assembly 100 at all. The anchor structure 106A may occlude the aneurysm (or any other vascular structure), for instance, by diverting the flow of blood.

The anchor structure may take on different configurations. For example, FIG. 2B shows a sensor system 150 being implanted into the aneurysm 20. The anchor structure 106B may take the form of a mesh or woven structure such as a basket. The anchor structure 106B may take the form of multiple loops that extend from the sensor system, e.g., two loops or three loops or four loops or more than four loops. Optionally, the multiple loops lie within a single plane. Optionally, the multiple loops are each of the same size. The anchor structure 106B may contact the wall of the aneurysm 20. FIG. 2C illustrates a sensor system 150 after it has been implanted in a bifurcation aneurysm 20 with the anchor structure 106C. The anchor structure 106C may include one or more coils to promote occlusion. After the sensor system 150 is implanted in the aneurysm 20, the blood can flow from a parent vessel 23 into associated daughter vessels 22 (as indicated by the arrows). The sensor system 150 can block blood from flowing into the aneurysm 20, which reduces the likelihood that the aneurysm 20 will grow.

FIG. 2D illustrates possible positioning of the sensor 102 or sensor assembly 100 within an aneurysm 20. The aneurysm 20 may have a neck 21, which can be located adjacent a parent artery 23. The aneurysm 20 may have a height H and the neck 21 may have a width W. The sensor 102 can be positioned at or near a middle of the height H of the aneurysm 20 and a middle of the width W of the neck 21. The positioning of the one or more sensors 102 or the sensor assembly 100 is important. For example, if the sensor 102 is positioned too far from the parent vessel 23, the sensor 102 may not be able to sense the blood flow. If the sensor 102 is positioned too close to the neck 21, the sensor 102 may cause unwanted blockages and other problems in treatment. Moreover, the sensor 102 may inadvertently flow out of the aneurysm 20 by, for example, the blood flow in the parent vessel 23.

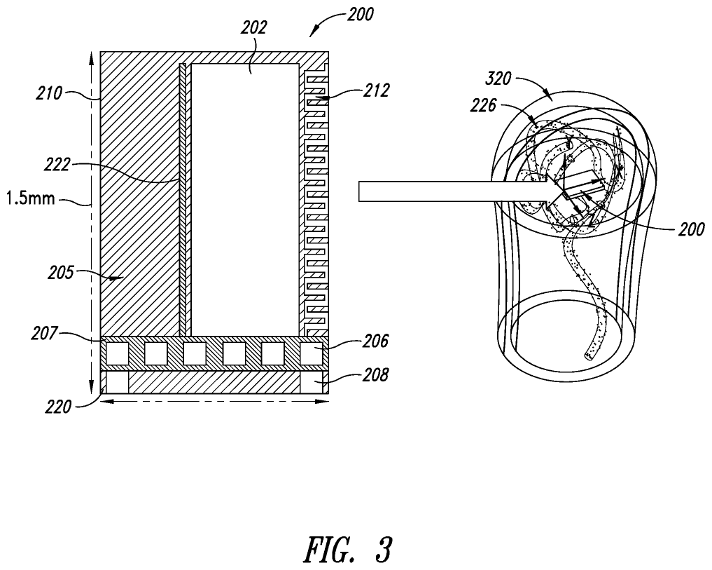

FIG. 3 illustrates a top view of the sensor assembly 200 (which can be similar to the sensor assembly 100 or any other sensor assembly described herein). The sensor assembly 200 can have a length and/or width that is less than or equal to about 1.5 mm or less than or equal to about 1.0 mm. A thickness of the sensor assembly 200 may be less than or equal to 500 microns or less than or equal to 300 microns.

The sensor assembly 200 can include a substrate layer (or substrate) 210, for example a silicon substrate, for attaching components. The substrate 210 can be a printed circuit board (PCB). The substrate 210 can be stacked on a power source (not shown).

The sensor assembly 200 can include one or more antennas 212 and one or more sensors 202 (e.g., a glucose sensor, oxygen sensor, metabolic sensor, motion sensors, or other sensor described herein). As illustrated, the sensor 202 and/or the antenna 212 may include platinum, a platinum alloy such as platinum/iridium alloy, gold, silver, or other suitable materials. Optionally, the sensor assembly 200 can include a reference electrode or balancer 222 to clean the signal. The reference electrode 222 can include silver or silver oxide. A region 205 of the substrate 210 may be kept clear for positioning of additional devices (such as, a communication circuitry, processor, etc.).

The sensor assembly 200 can include one or more contact pads 206 for component linking. The contact pads 206 may be carried by an insulation layer. The sensor assembly 200 can include one or more ground pads 208 for circuit conduit and switch interaction. The ground pads 208 may be carried by the power source. As illustrated, the contact pads 206 may be positioned between the ground pads 208 and the sensor 102.

The sensor assembly 200 is illustrated as being implanted in an aneurysm 320 (which can be similar to the aneurysm 20), such as being surrounded by the anchoring structure 226 (shown as coils).

### A. Antenna(s)

As previously described, the sensor assembly 100 (or 200 or any other sensor assembly disclosed herein) can include one or more antennas, such as the antenna 112. The antenna 112 may be in electrical communication with the one or more other components of the sensor assembly 100, for example a communication circuitry (such as, a transceiver) and/or charging circuitry (such as, for inductive charging). The antenna 112 can transmit sensor data or other data related to the sensor assembly 100 to a receiver (e.g., a hub within the body and/or a base station or other computing device outside the body), which may also transmit data, such as one or more commands, and can be referred to as receiver and transmitter. The receiver can be integrated into an article of clothing worn by the patient. For example, in case of the sensor assembly being implanted into an intracranial aneurysm, the receiver can be integrated, for instance, into a hat, headband, or scarf worn by the patient or more generally into any garment or clothing that can be worn by the patient in the area from the upper torso to the cranium. The antenna 112 can continuously transmit sensor data or intermittently transmit sensor data at predetermined time intervals or upon receipt of a command from the receiver. The antenna 112 may allow the sensor assembly 100 to communicate with a base station configured for use with the sensor assembly 100. The antenna 112 can be configured to support one or more of Bluetooth™, Bluetooth® Low Energy (BTLE), WiFi®, MICS, Industrial Scientific, and Medical (ISM), or Zigbee protocols.

The antenna 112 can include a filter (not shown). The filter can be any suitable bandpass filter, such as a surface acoustic wave (“SAW”) filter or a bulk acoustic wave (“BAW”), passive element(s) (such as, one or more inductors or capacitors), or microstrip elements in the PCB. The antenna 112 can be suitable for the frequency band in which the RF transceiver and/or the antenna 112 generates signals for transmission by the antenna 112, and for the frequency band in which a base station generates signals for reception by the antenna 112.

The antenna 112 may be constructed from one or more materials (e.g., pure or alloy material). For example, the antenna 112 can be constructed from one or more of the following: platinum iridium, platinum, gold, or a coated shape memory wire. The antenna 112 can be constructed from etching a biocompatible metal onto a biocompatible polymer. The coated shape memory wire can include an inner nitinol wire (or a wire made from another shape memory alloy or a wire made from multiple shape memory alloys) coated and/or plated with a conductive material so that the antenna 112 operates in the frequency range of interest. The one or more materials can have a conductivity of between 1.0−6.0×10⁶ S/M. The one or more materials can be adapted to interact with or be neutral to the sensor 102, as the sensor 102 interacts with a treatment zone.

The antenna 112 can be constructed from a wire (such as, platinum iridium, platinum, gold, or nitinol wire) or have a tubular construction (such as, a coated wire). In one embodiment the antenna is formed in part or wholly from platinum and iridium such as a platinum/iridium alloy, e.g., an approximately 80/20 platinum/iridium alloy, e.g., about 75-85 parts platinum and about 15-25 parts iridium, or an approximately 90/10 platinum/iridium alloy, e.g., about 85-95 parts platinum and about 5-15 parts iridium. In one embodiment the antenna is formed from a gold outer cladding with nitinol inside. The tubular configuration can contemplate the use of multiple materials for constructing the antenna 112. For instance, a first material (such as, nitinol or another shape memory alloy) can be utilized for shape setting and to facilitate compression and expansion of the antenna 112. The second material can be utilized for wireless transmission and reception by the antenna 112. The second material can be platinum iridium, platinum, gold, copper, or the like. In some cases, the second material can be also used for the sensing, as described herein in connection with sensing using a chemical reaction. The second material can be placed on top of or at least partially enclose the first material.

The antenna 112 can be sufficiently flexible, pliable, and/or conformable such that the sensor assembly 100 (including the antenna) can transition between a compressed configuration (for loading into the delivery system) and an expanded configuration (when inserted into or positioned at a treatment site or zone). For example, the antenna 112 can be compressed for loading into the delivery system and expanded upon release from the delivery system. In the expanded configuration, the antenna 112 may provide stabilizing or anchoring functionality for the aneurysm and/or other component(s) of the sensor assembly 100. The antenna 112 can include a flexible parylene antenna. The one or more antennas 112 can be compressed into the compressed configuration and loaded into a delivery system. Once the antenna 112 is implanted into the patient, the antenna 112 may expand into the expanded configuration. In such configuration, the antenna 112 can anchor the sensor assembly 100 within the patient (e.g., in or near a treatment zone). In the expanded configuration, the antenna 112 can be sized and adapted to conform to the inner surface of or near the treatment zone (e.g., the inner surface of an aneurysm).

The antenna 112 can form the anchoring structure for the sensor system 150. The antenna 112 can include a single axis loop, a dual axis loop, or a spherical loop to enhance the capability of contact with the vascular structure or other anchoring structure. The antenna can locate the sensor assembly 100 in the patient (e.g., a position about the aneurysm neck entrance). When the sensor assembly 100 is implanted in a patient, the antenna 112 can, for example, interact with an aneurysm wall and act as an anchor contact with the aneurysm to maintain a position of the sensor assembly 100 for adequate monitoring of the fluid exchange between the aneurysm and the associated parent artery. For example, the antenna 112 may include one or more prongs and/or prong extensions that can interact with the inner wall of the aneurysm. The one or more prongs and/or prong extensions can rounded ends such that the prongs and/or prong extensions are atraumatic. As described herein, a separate anchoring structure 106 can be used to support the sensor assembly 100 in the treatment site. In those configurations, the separate anchoring structure 106 can directly or indirectly contact or couple to the antenna 112. The antenna 112 can be deployed such that the antenna does not obstruct the implantation of the separate anchoring structure 106. The separate anchoring structure 106 can conform to the inner surface of or near the treatment zone.

The antenna 112 or other component of the sensor assembly 100 may be manufactured to carry a material that complements or inhibits the interaction desired by the treatment method, as further described below with respect to the one or more sensor(s). For example, the antenna 112 can be coated with the degradable material on the outside or inside of the antenna(s) 112, which can release some of the material or other byproducts of the material's degradation into the treatment site. The antenna 112 and/or any other component of the sensor assembly 100 can be coated with conformal coating (such as, parylene, silicone, conformal epoxy, etc.). For example, the materials or other byproducts may be released based on a conductivity switch, which is further described below in relation to the Sensor System. In some cases, the antenna 112 and/or any other component of the sensor assembly 100 can be coated with material(s) and/or drug(s) that promote clotting (such as, coagulant) or otherwise promote closure. In some implementations, the antenna 112 and/or any other component of the sensor assembly 100 can be coated with material(s) and/or drug(s) that inhibit clotting (such as, anticoagulant) or otherwise inhibit closure. The antenna 112 can act as a coil implant for the sensor 102 and work in conjunction with the coils or spherical implants to fill and obstruct flow into and about the aneurysm zone. The antenna 112 can serve as supporting structure for the treatment site (for instance, an aneurysm).

With reference to FIGS. 2A-2D and FIG. 3, it is not trivial to design an antenna for implanting into the treatment site. In some cases, the antenna needs to be small. For instance, the antenna may contour to a cranial aneurysm, which can have a radius of about 2.5 mm (or less) to about 10 mm (or more). As disclosed herein, the antenna may have different three-dimensional configurations to contour to or comply with the aneurysm wall. In some implementations, the antenna may be compressible and expandable, operate in a desired frequency band, and transmit and receive data over a desired distance In case of a cranial aneurysm, such distance can be, for instance, at least 10 cm (or less) in any direction, at least 11 cm in any direction, at least 12 cm in any direction, at least 13 cm in any direction, at least 14 cm in any direction, or at least 15 cm (or more) in any direction. The distance can be relatively short since the receiver can be positioned close to the treatment site, such as supported by a hat, headband, or scarf as described herein. Some of the above design parameters may be conflicting. For instance, decreasing the size of the antenna increases the frequency range of operation, but the increased frequency (and shorter wavelength) would cause more loss in the tissue due to increased scattering. As another example, decreasing the frequency range of the antenna would increase the operational distance, but would make the antenna larger.

Designing the antenna can involve creating a model of the treatment site and surrounding tissue, anchor structure, sensor, and antenna. For a cranial aneurysm (which will be used as an example throughout this section), the model can include a vessel wall, anchor structure (such as, coil of varying density), sensor substrate (for instance, generic dielectric, such as FR4, Rogers, or Isola), and different antenna configurations. An electromagnetic (EM) simulator (such as, a high frequency EM solver) could be utilized to create and run the model. Realized antenna gains, reflection coefficient indicating how much power is reflected from the antenna (which is related to the return loss and can be expressed by the S11 parameter), and impedance and radiation field patterns can be analyzed for each permutation and variation of input variables (such as, frequency and communication transmission). Modeling can be used to select the best suitable antenna design for the desired operating distance.

The following one or more assumptions can be made for the modeling. It may be assumed that there is at least one electrical connection between the antenna and the rest of the electronic circuitry of the sensor assembly (such as, the connection between the antenna and transceiver). It may be assumed that there is at least one connection from the sensor assembly to the anchor structure (for grounding). It may be assumed that the antenna is made up of a continuous wire, which can be shaped in a conical or sinusoidal wrap pattern on a flat two-dimensional surface. The antenna can be set to a spherical or substantially spherical shape to comply with the anatomical placement in the aneurysm (such as, to contour to the anatomy).

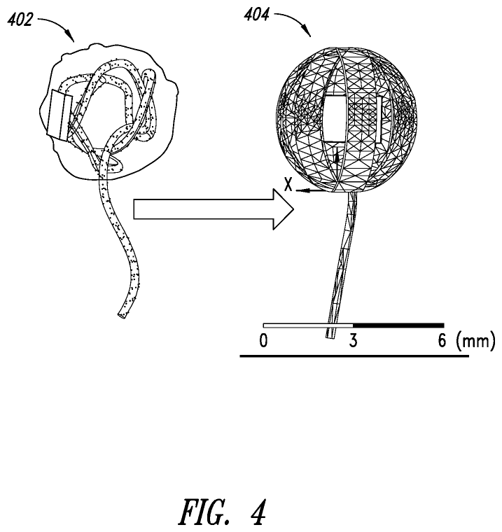

In some cases, it may be advantageous to make one or more simplifications during the modeling. For example, to speed up the simulation (for example, to reduce the simulation from several days or more to several hours or less), a dense coil anchor structure 402 can be simplified to a flatter structure 404 as illustrated in FIG. 4.

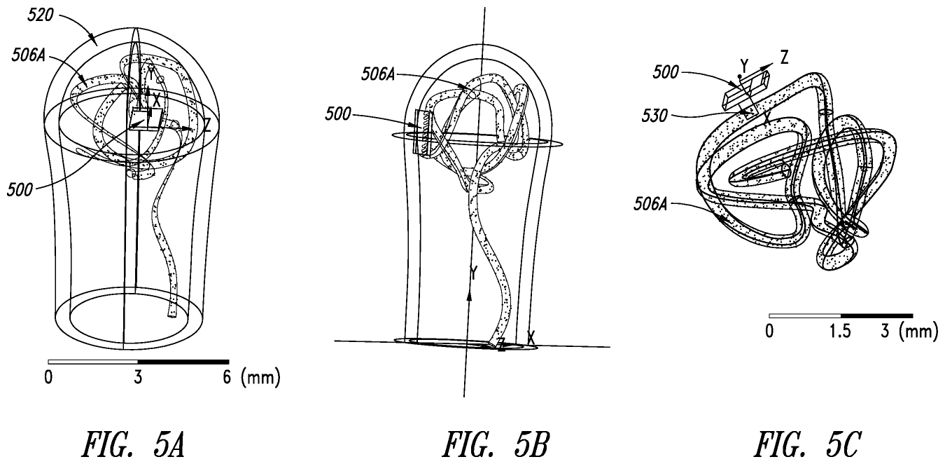

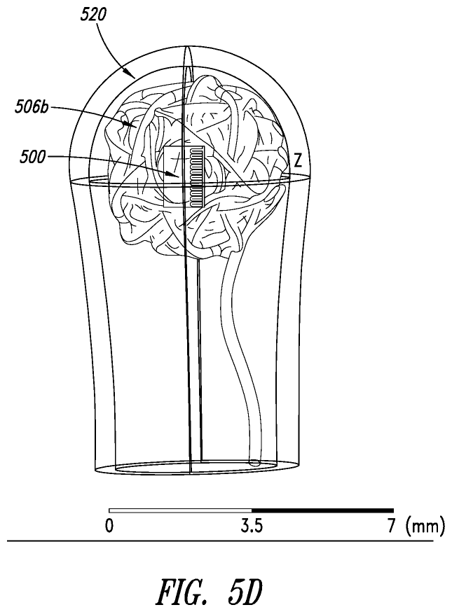

One antenna design that had been modeled includes a meander (or meandered) monopole antenna (such as, the antenna 212 illustrated in FIG. 3). FIG. 5A illustrates a sensor assembly 500 (which can be similar to the sensor assembly 200) with a meandered monopole antenna. The sensor assembly 500 is illustrated as having been implanted in an aneurysm 520. The sensor assembly 500 is positioned internal to an anchor structure 506A (such as, being surrounded by the loosely coupled coils). In some cases, modeling such configuration can demonstrate unacceptable performance of the antenna. For example, peak gain at 2.45 GHZ (which corresponds to the Bluetooth band, ISM band (also used by Zigbee), or WLAN band) can be around −64 decibel (dB) and at 403 MHZ (which corresponds to the MICS band with frequency range of 402 to 405 MHz) can be around −114 dB, both of which are far too low. As another example, the impedance of the antenna may be far outside the Smith chart, which would be quite challenging to match to the input impedance of the transceiver (for instance, 50 Ohm). These shortcomings may be due to the conductive coil structure surrounding the antenna and acting as a Faraday shield or cage for the antenna.

FIG. 5B illustrates positioning the sensor assembly 500 outside of the anchor structure 506A (such as, external to the coil structure). In some cases, modeling such configuration can demonstrate unacceptable performance of the antenna and little advantage over the configuration in FIG. 5A.

FIG. 5C illustrates positioning the sensor assembly 500 outside of the anchor structure 506A (as in FIG. 5B) and grounding the ground of the sensor assembly substrate (such as, a printed circuit board) to the anchor structure 506A (for instance, with a connection 530). In this configuration, the antenna is grounded to the anchor structure 506A, which can improve the antenna's performance. In some cases, modeling such configuration can demonstrate improvements in the peak gains. For instance, the peak gain at 2.45 GHz can be about −53 dB (or improved by about 11 dB as compared to FIG. 5A), and the peak gain at 403 MHz can be about −88 dB (or improved by about 26 dB as compared to FIG. 5A). This can demonstrate that grounding can be important to achieve the desired performance by the antenna.

FIG. 5D illustrates positioning the sensor assembly 500 outside of the anchor structure 506B and grounding the sensor assembly to the anchor structure (as in FIG. 5C). The anchor structure 506B can be more dense than the structure 506A. The anchor structure 506B can represent densely coupled coils 506B (which may be a possible configuration depending on the aneurysm being treated). In some cases, modeling such configuration can show that the addition of coils does not affect the peak gains or change the impedance of the antenna, which indicates that the performance of the antenna does not degrade (as compared to FIG. 5C). Modeling can show that the radiation pattern of the antenna can be smoother, which can be advantageous.

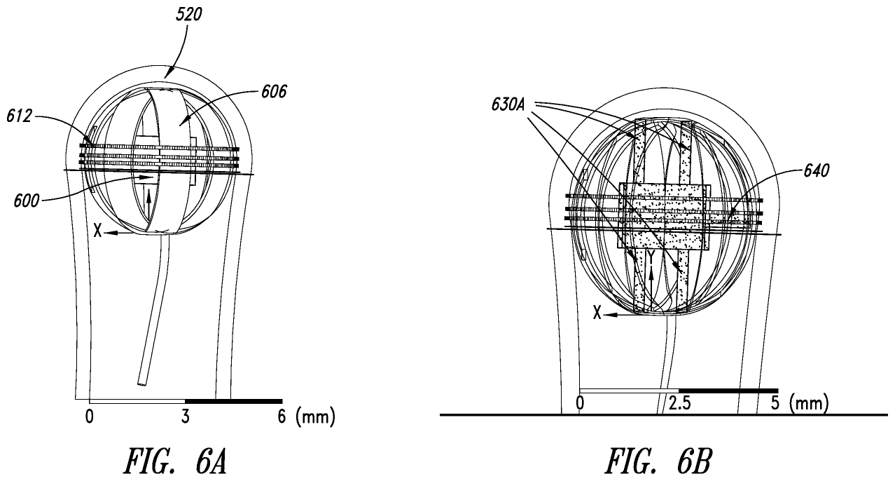

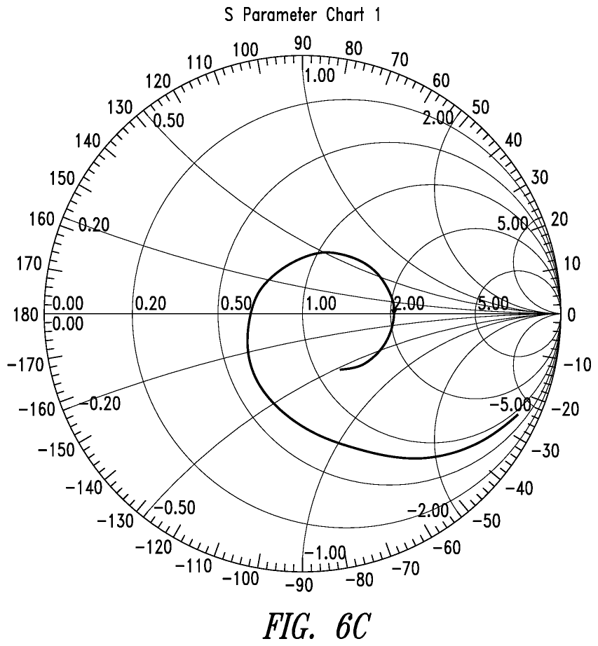

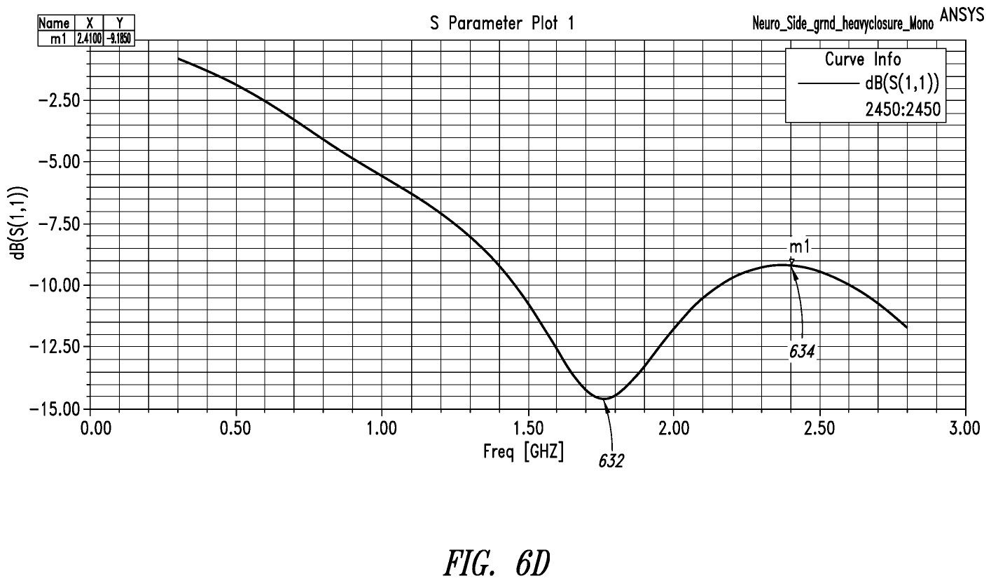

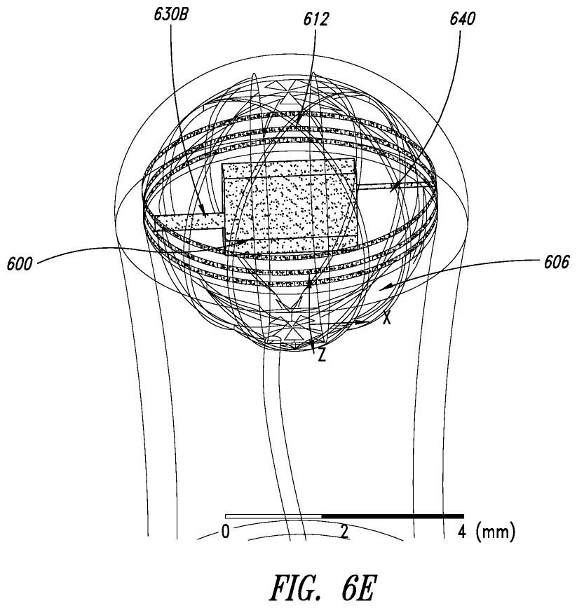

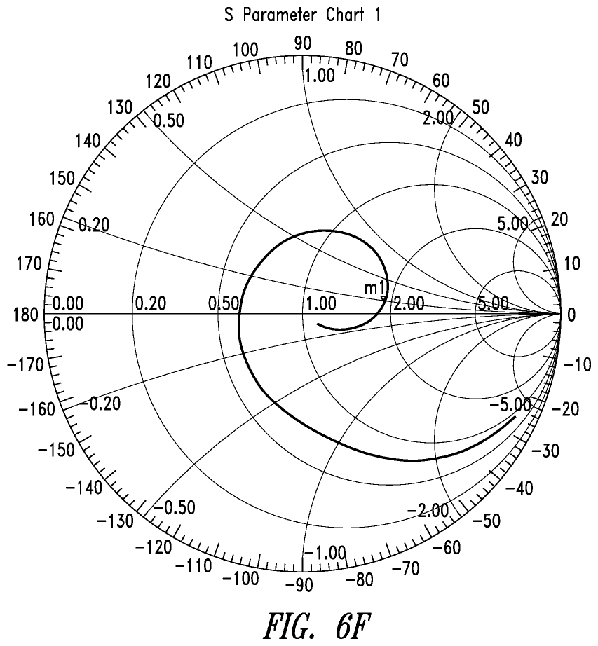

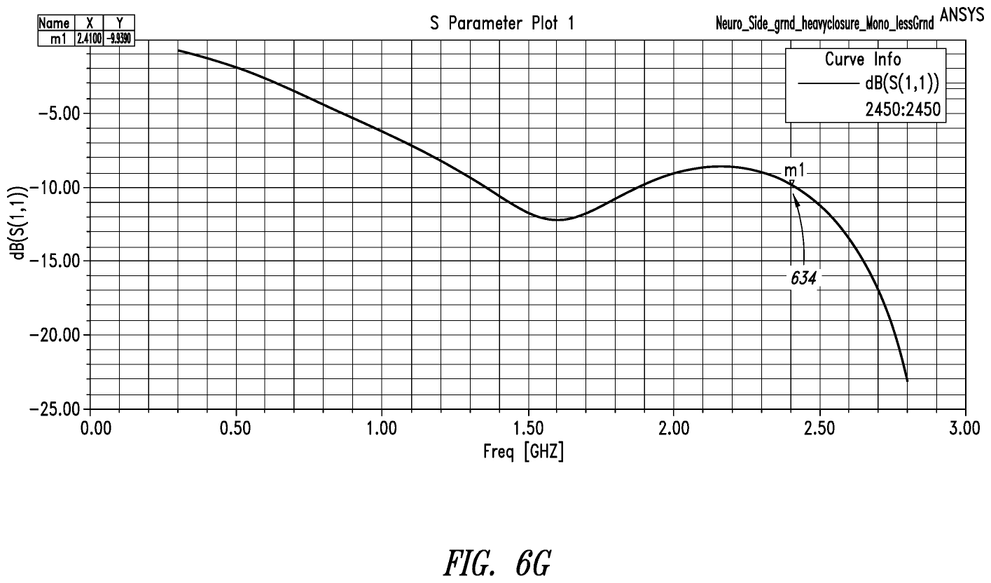

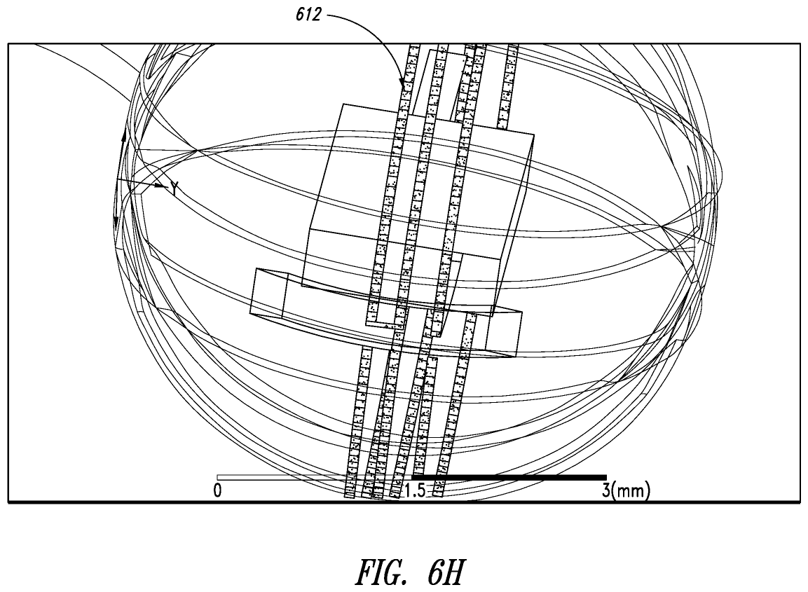

Another antenna design that had been modeled includes a flat monopole antenna, which can be positioned around a three-dimensional structure (such as, a three-dimensional anchor structure, which can be spherical or substantially spherical to match the anatomy of the aneurysm). This type of antenna should be distinguished from a helical (or helix) or loop antenna. Unlike the helical antenna, a flat monopole antenna in two-dimensional configuration can be a straight wire that is meandered (or continuously wound back and forth, but does not wind back on itself). Such flat monopole antenna 612 is illustrated in FIG. 6H. Unlike the meandered monopole antenna described in connection with FIGS. 5A to 5D, the flat monopole antenna 612 can be positioned around a three-dimensional structure as is illustrated and described below.

FIG. 6A illustrates a sensor assembly 600 (which can be similar to the sensor assembly 200) with the flat monopole antenna 612 positioned around a spherical anchor structure 606 (such as, a coil structure, for instance, similar to 404). While the sensor assembly 600 is positioned internal to the anchor structure 606, the antenna 612 is positioned external to the anchor structure. The anchor structure 606 can represent densely coupled coils (such as, 506B in FIG. 5D). As shown in FIG. 6B, the ground of a substrate of the sensor assembly 600 can be grounded to the anchor structure 606 with a plurality of connections 630A (such as, four connections). In some cases, modeling such configuration can demonstrate improved (and possibly acceptable performance) of the antenna 612. For example, peak gain at 2.45 GHz can be around −27 dB and at 403 MHz can be around −71 dB, which represent improvements over the configurations in FIGS. 5A to 5D (with the improvement in the Bluetooth or ISM band being particularly significant).

As another example, the impedance of the antenna can be close to being matched (without using any matching circuitry). This is illustrated in FIG. 6C, which depicts impedance of the antenna 612 determined for the range of frequencies in the Bluetooth frequency band (2400 to 2483.5 MHz) or the ISM frequency band (2.4 to 2.5 GHZ) and plotted on a Smith chart (center point of the Smith chart labeled “1.0” can correspond to the desired impedance of 50 Ohm). The S11 parameter of the antenna 612 across the range of frequencies in the frequency band is illustrated in FIG. 6D. Point 634 depicts that at 2.45 GHZ, the S11 value is between-9 dB and −9.5 dB, representing good performance (since, in some cases, the target S11 value can be −5 dB or less or preferably-10 dB or less). The resonant frequency of the antenna 612 (indicated by the trough 632 in FIG. 6D) is near the desired frequency band (2400 to 2483.5 MHZ). Suitable matching circuitry can be used to shift the resonant frequency of the antenna 612 to the desired frequency band and to match the impedance of the transceiver (such as, 50 Ohm). The matching circuitry can be interposed between the antenna and the transceiver (for instance, connected to an antenna feed 640, which can serve as an electric connection between the antenna a transceiver of the sensor assembly 600).

FIG. 6E illustrates the same structure as in FIG. 6A, but with a single ground connection 630B between the ground of the substrate of the sensor assembly 600 and the anchor structure 606. It may be advantageous to reduce the number of ground connections, for instance, to facilitate insertion of the sensor assembly 600, antenna 612, and the anchor structure 606 into an aneurysm using a delivery system (such as, a catheter). In some cases, the choice and configuration (such as, density) of the anchor structure would be selected by the surgeon, and it may not be possible to achieve a plurality of ground connections. In some cases, modeling the configuration shown in FIG. 6D can demonstrate negligible difference as compared to modeling the configuration shown in FIG. 6A. For example, peak gain at 2.45 GHz can be around −28 dB (as compared to −27 dB for the configuration of FIG. 6A). As illustrated by the Smith chart in FIG. 6F, the impedance of the antenna 612 has shifted slightly (as compared with FIG. 6C), but still remains close to being matched. As illustrated by the S11 plot in FIG. 6G, the S11 value at 2.45 GHz is −10 dB (point 634).

Antenna 612 can be oriented in any plane to facilitate implantation into the treatment site by a delivery system. As described herein, the antenna 612 can function as a supporting structure for the aneurysm.

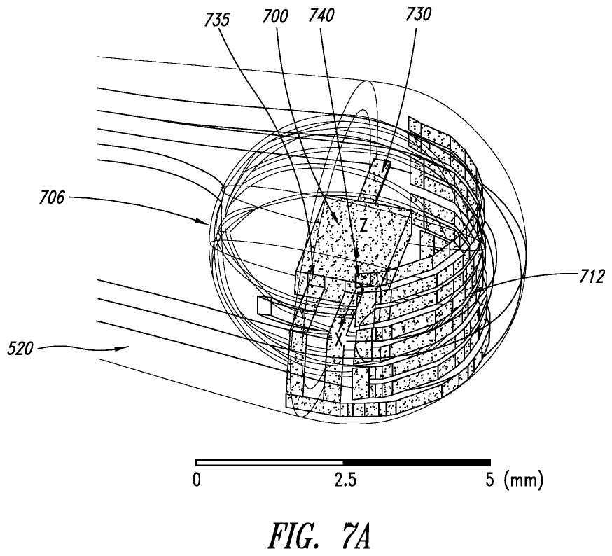

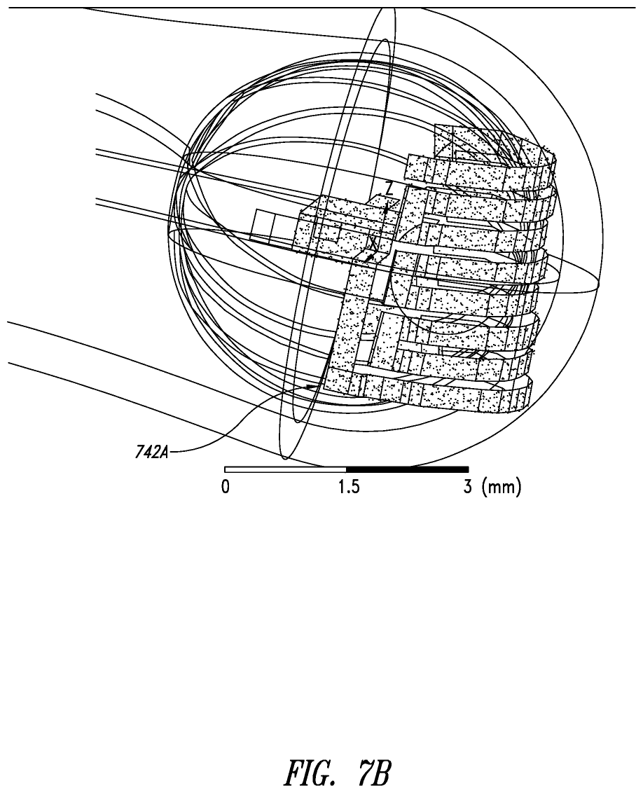

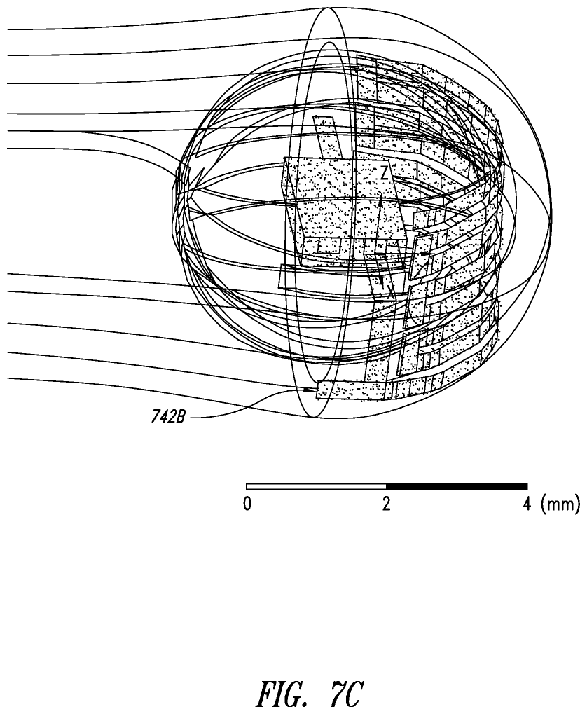

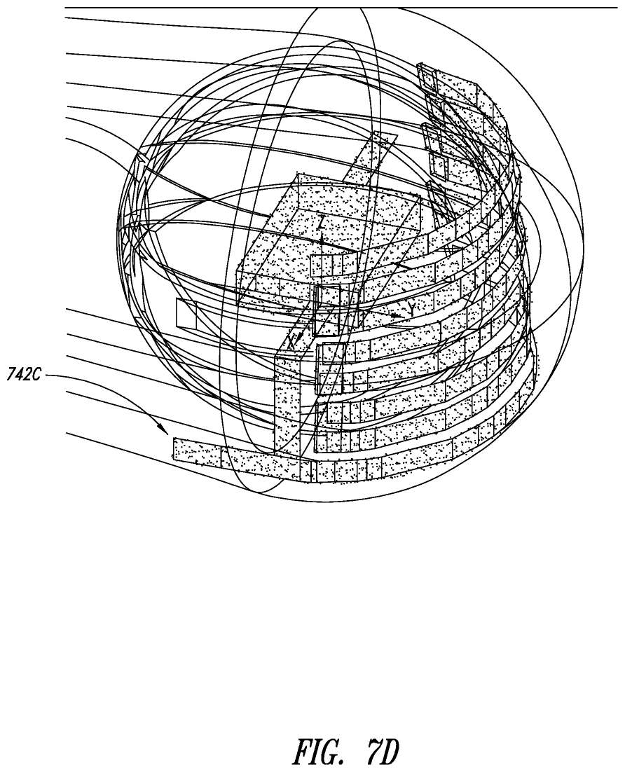

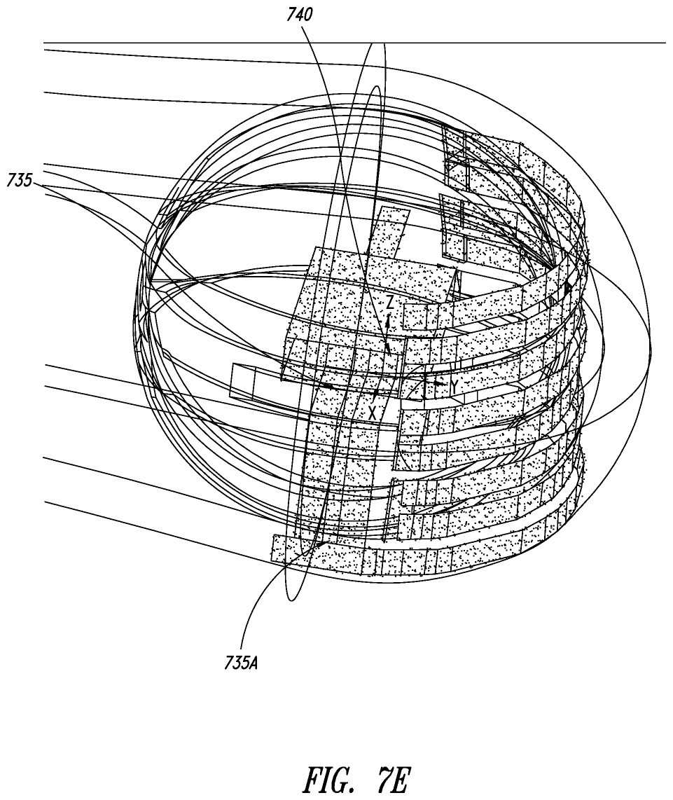

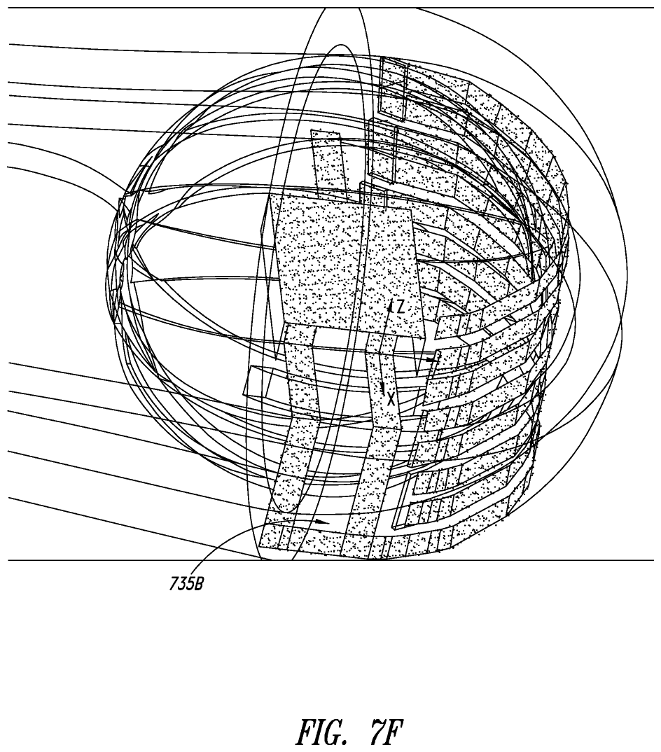

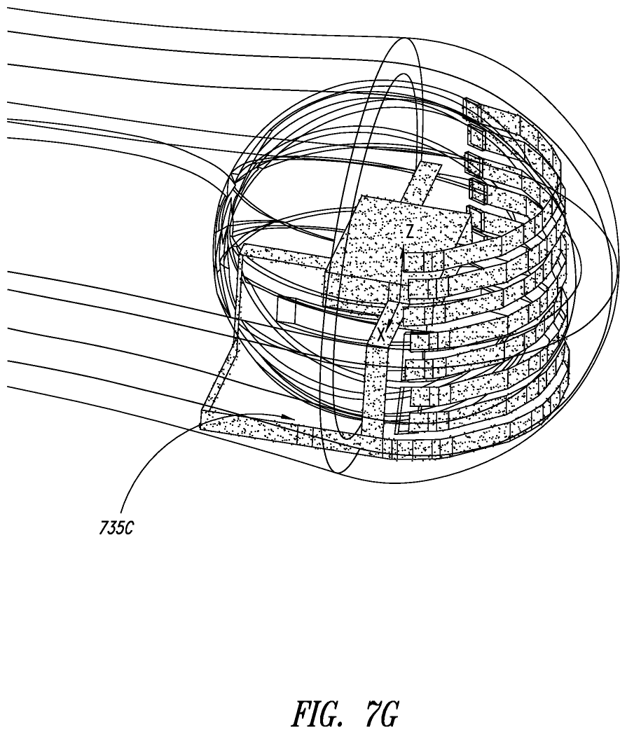

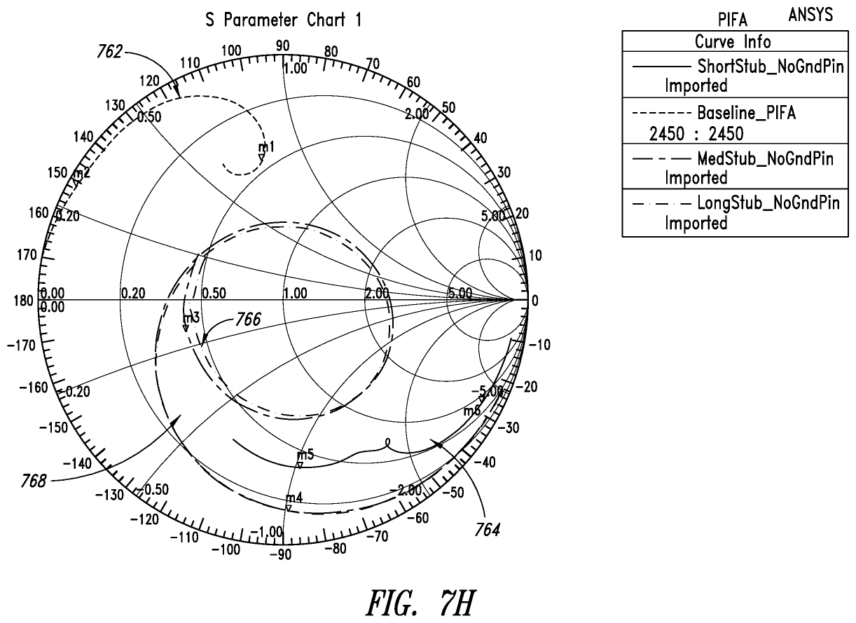

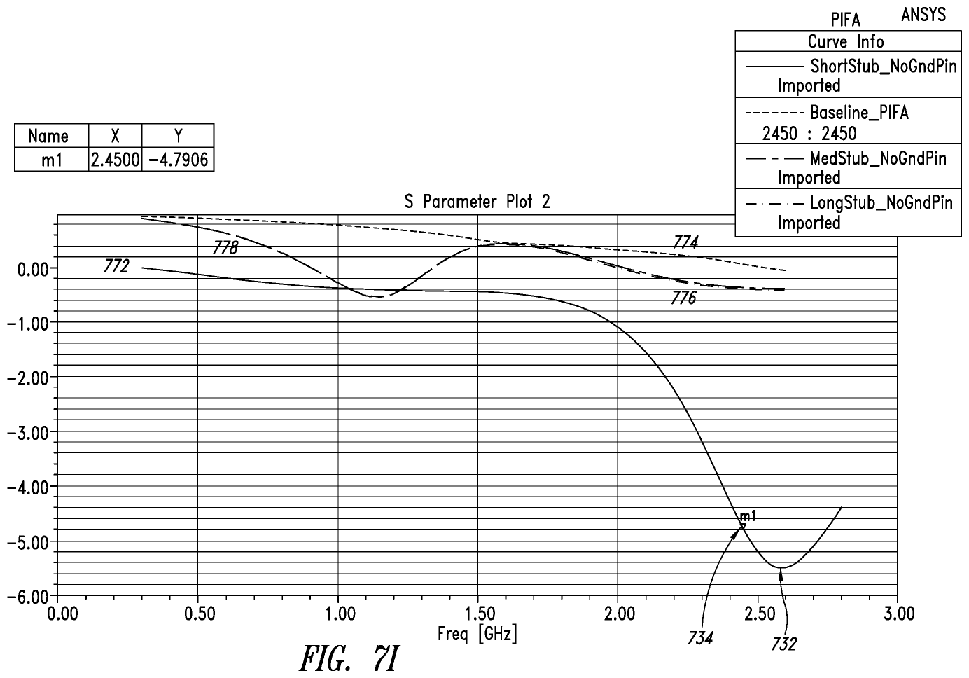

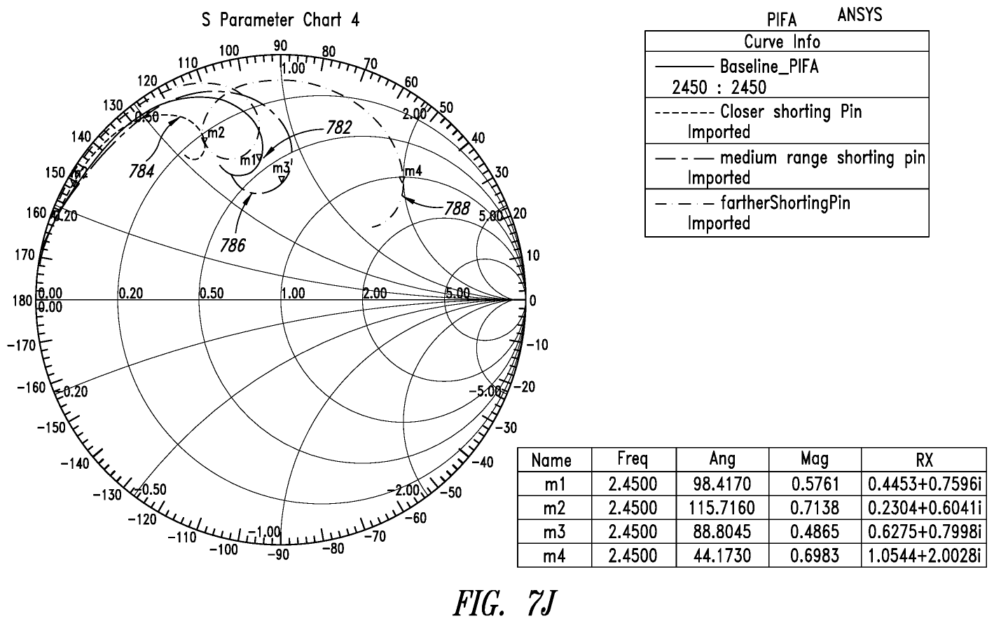

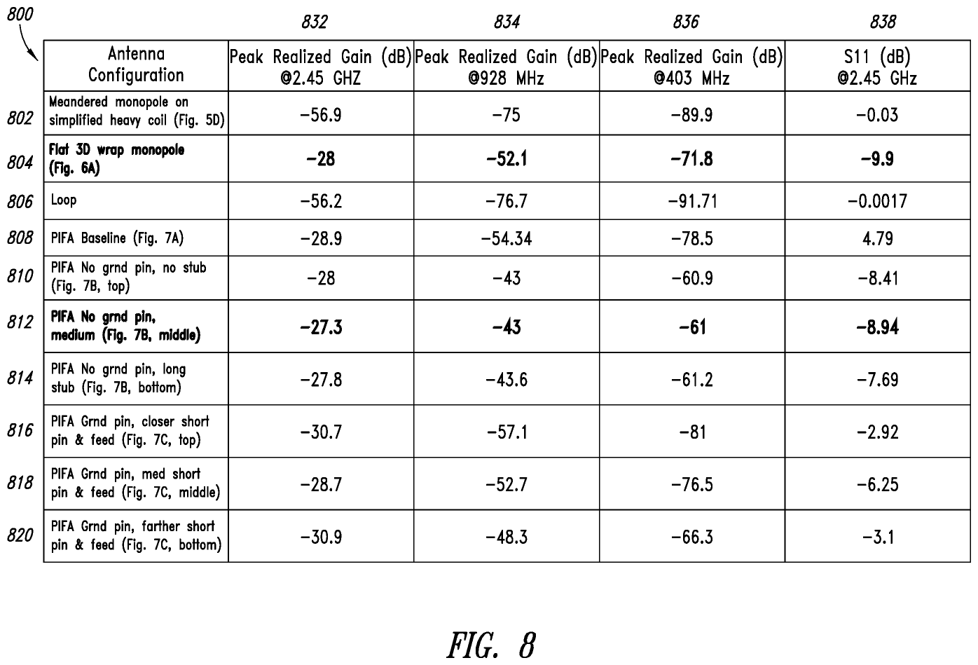

Another antenna design that had been modeled includes a planar inverted F-antenna (PIFA). A PIFA antenna can be a rectangular (or square) patch antenna that includes an antenna, ground plane, substrate (positioned between the antenna and ground plane), feed, and shortening pin (or plane) connecting the antenna to the ground plane. The shortening pin can be positioned at or proximate to the feed of the antenna. FIG. 7A illustrates a sensor assembly 700 (which can be similar to the sensor assembly 200) with a PIFA antenna 712 positioned around a spherical anchor structure 706 (such as, a coil structure, for instance, similar to 404). While the sensor assembly 700 is positioned internal to the anchor structure 706, the antenna 712 is positioned external to the anchor structure. The anchor structure 706 can represent densely coupled coils (such as, 506B in FIG. 5D). Unlike certain PIFA antennas, the antenna 712 may not include a substrate, which can allow the antenna to be compressed and expanded. Antenna feed is shown as 740. Antenna ground connection (or ground pin) is shown as 735. The ground connection 735 can correspond to the shortening pin. A single ground connection 730 between the ground of a substrate of the sensor assembly 700 and the anchor structure 706 is illustrated. In some cases, multiple ground connections can be used, as described herein. In some cases, modeling the configuration illustrated in FIG. 7A can demonstrate improved (and possibly acceptable performance) of the antenna 712. For example, peak gain at 2.45 GHz can be around −29 dB and at 403 MHz can be around −79 dB, which represent improvements over the configurations in FIGS. 5A to 5D (with the improvement in the Bluetooth or ISM band being particularly significant).

PIFA antenna can be tuned by varying one or more properties of the feed or ground connections. This can change one or more of the resonant frequency, capacitance, or inductance of the antenna 712. Tuning the antenna can improve performance and require less circuit elements for the matching circuitry (or, in some cases, no matching circuitry at all). Advantageously, this can save space and conserve power.

With reference to FIG. 7B, FIG. 7C and FIG. 7D, ground pin 735 can be left floating (instead of being connected to the sensor assembly 700) and its length can be varied, which can change the inductance of the antenna 712. This can be referred to as varying the length of a stub. FIG. 7B illustrates no stub 742A. FIG. 7C illustrates a short stub 742B. FIG. 7D illustrates a long stub 742C. While a medium stub is not illustrated, this variation can also be taken into account. With reference to FIG. 7E, FIG. 7F and FIG. 7G, the distance between the feed 740 and the ground pin 735 (see FIG. 7E) can be varied, which can change the capacitance of the antenna 712. FIG. 7E illustrates a short distance 735A (in comparison with the reference design in FIG. 7A). FIG. 7F illustrates a medium distance 735B. FIG. 7G illustrates a long distance 735C.

The impedance of the antenna 712 can be close to being matched (without using any matching circuitry). This is illustrated in FIG. 7H, which depicts impedance of the antenna 712 determined for the range of frequencies in the Bluetooth frequency band (2400 to 2483.5 MHz) or ISM frequency band (2.4 to 2.5 GHZ) and plotted on a Smith chart. Plot 762 can correspond to the reference (or baseline) configuration illustrated in FIG. 7A. Plot 764 can illustrate a short stub (such as, 742B in FIG. 7C). Plot 766 can illustrate a medium stub (not shown in any of FIG. 7B, FIG. 7C or FIG. 7D). Plot 768 can illustrate a long stub (such as, 742C in FIG. 7D).

The antenna 712 can exhibit good return loss. FIG. 7I (number 7, letter I) illustrates the S11 parameter of the antenna 712 across a range of frequencies in the frequency band of interest. Plot 772 can correspond to the reference configuration illustrated in FIG. 7A. Plot 774 can correspond to the short stub. Plot 774 can correspond to the medium stub. Plot 778 can correspond to the long stub. As illustrated by the plot 772, reference configuration (illustrated in FIG. 7A and in FIG. 7B) has S11 value of almost-5 dB at 2.45 GHZ (illustrated by 734) and has a resonant frequency (indicated by the through 732) that is near the desired frequency band (of 2400 to 2483.5 MHz).

FIG. 7J illustrates impedance of the antenna 712 determined for the range of frequencies in the Bluetooth frequency band or ISM frequency band and plotted on the Smith chart. Plot 782 can correspond to the reference (or baseline) configuration illustrated in FIG. 7A. Plot 784 can illustrate a short distance between the feed 740 and the ground pin 735 (such as, 735A in FIG. 7E). Plot 786 can illustrate a medium distance (such as, 735B in FIG. 7F). Plot 788 can illustrate a long distance (such as, 735C in FIG. 7G). Change in the capacitance as a result of the medium distance configuration (plot 786) illustrates better impedance matching as the antenna impedance is closer to the center point (which indicates the desired match). S11 value of this configuration at 2.45 GHz can be about-9 dB, which can indicate good performance of the antenna.

As explained above, the impedance can slightly shift when a single ground connection 730 is used. As described herein, suitable matching circuitry can be used to shift the resonant frequency of the antenna 712 to the desired frequency band and to match the impedance of the transceiver (such as, 50 Ohm).

Antenna 712 can be oriented in any plane to facilitate implantation into the treatment site by a delivery system.

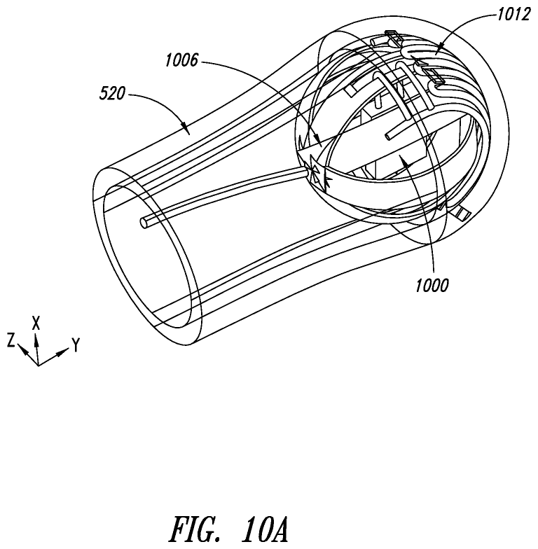

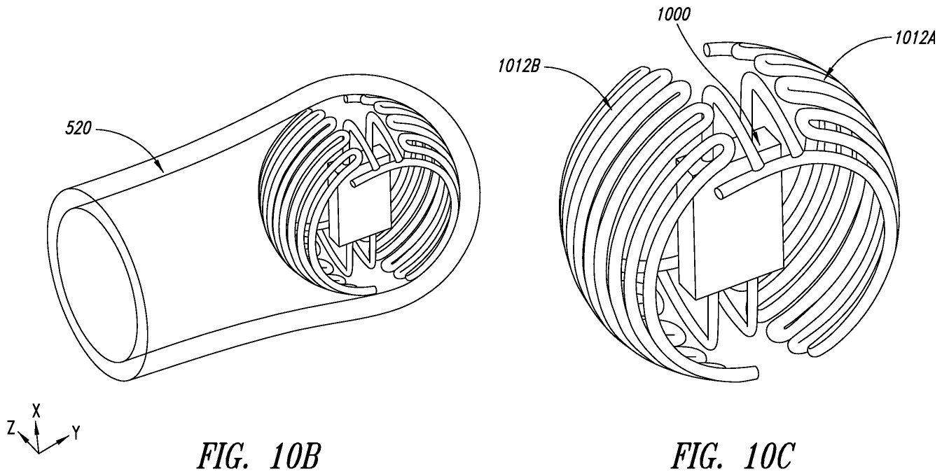

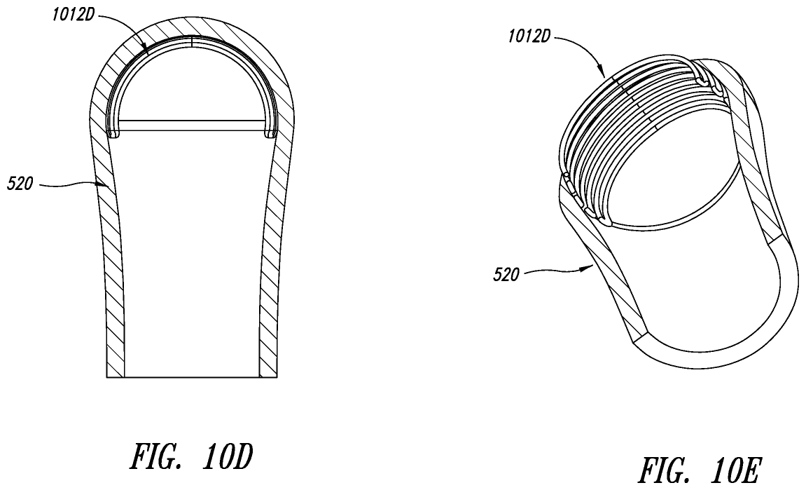

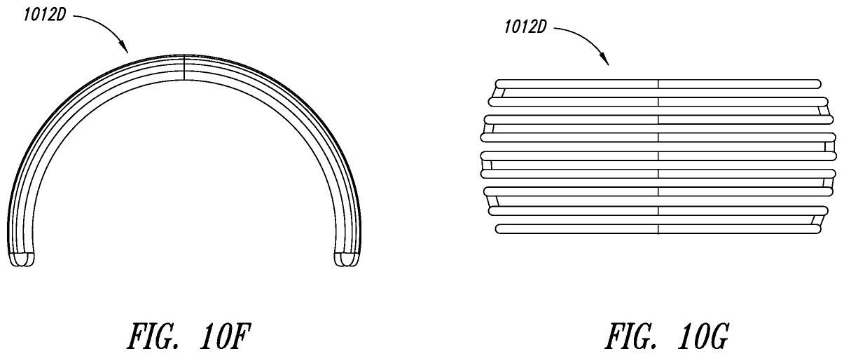

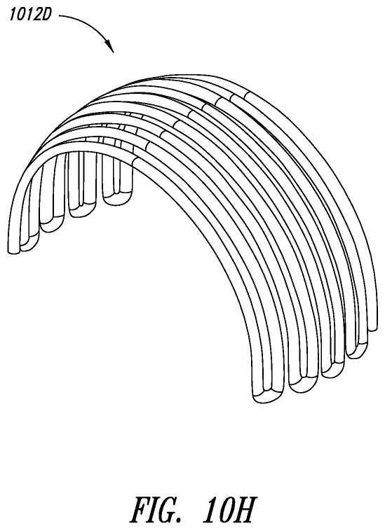

Antenna 712 (or any of the other antennas disclosed herein, such as the antenna 612) can be positioned in or proximal to the tip of the aneurysm 520. The antenna 712 can conform to the shape of the aneurysm. For example, the antenna can form a semi-spherical or substantially semi-spherical shape. Advantageously, the antenna 712 can provide supporting structure for the aneurysm. For example, the antenna 712 can conform to the aneurysm and provide minimal radial outward opposing force to the wall(s) of the aneurysm. The antenna 712 can comply with (or contour to) the collapsing wall(s) as the treatment halts blood flow into and pressurize the aneurysm. This is illustrated, for example, in FIGS. 10D and 10E. In some implementations, antenna 712 (or any of the other antennas disclosed herein, such as the antenna 612) can be positioned in the middle or bottom of the aneurysm 520. For example, the antenna can be positioned at the bottom of the aneurysm (or proximal to the neck) to occlude the aneurysm, for example, by diverting the flow of blood.

FIG. 8 provides a table 800 summarizing performance of various antenna configurations as determined with the modeling. In some cases, data provided in the table 800 has been obtained when all configurations included a single ground connection between the ground of a substrate of a sensor assembly and the anchor structure. Column 832 provides peak gain (in dB) at 2.45 GHZ, which can correspond to the Bluetooth or ISM band. Column 834 provides peak gain (in dB) at 928 MHz, which can correspond to another unlicensed frequency band. Column 836 provides peak gain (in dB) at 403 MHZ, which can correspond to the MICS band. Column 836 provides the S11 value (in dB) at 2.45 GHz.

Row 802 lists performance of the configuration illustrated in FIG. 5D (meandered monopole antenna enclosed by a dense coil structure). Row 804 lists performance of the configuration illustrated in FIG. 6A (flat monopole antenna positioned around a three-dimensional structure). Row 806 lists performance of a sensor assembly with a loop antenna (not illustrated in the drawings). Row 808 lists performance of the configuration illustrated in FIG. 7A (baseline PIFA configuration). Row 810 lists performance of the configuration illustrated in FIG. 7B (no ground pin, no stub). Row 812 lists performance of the configuration illustrated in FIG. 7C (no ground pin, medium stub). Row 814 lists performance of the configuration illustrated in FIG. 7D (no ground pin, long stub). Row 816 lists performance of the configuration illustrated in FIG. 7E (short distance between the ground pin and antenna feed). Row 818 lists performance of the configuration illustrated in FIG. 7F (medium distance between the ground pin and antenna feed). Row 820 lists performance of the configuration illustrated in FIG. 7G (long distance between the ground pin and antenna feed). The configurations in rows 804 (flat monopole antenna) and 812 (PIFA antenna with medium stub) exhibit good performance for peak gain and S11 value at 2.45 GHz.

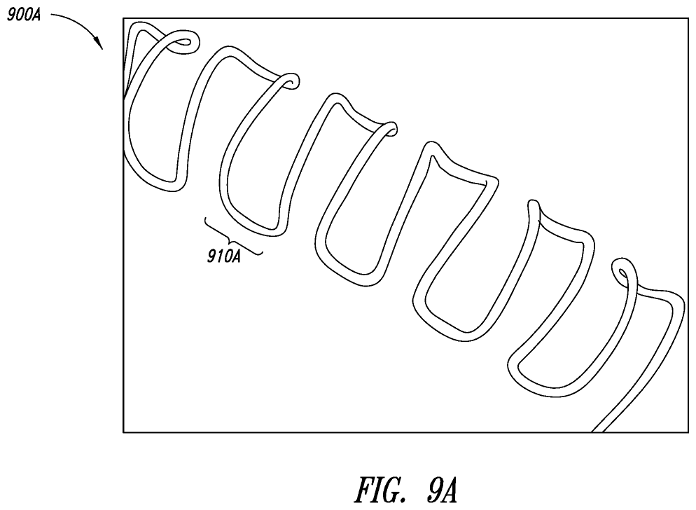

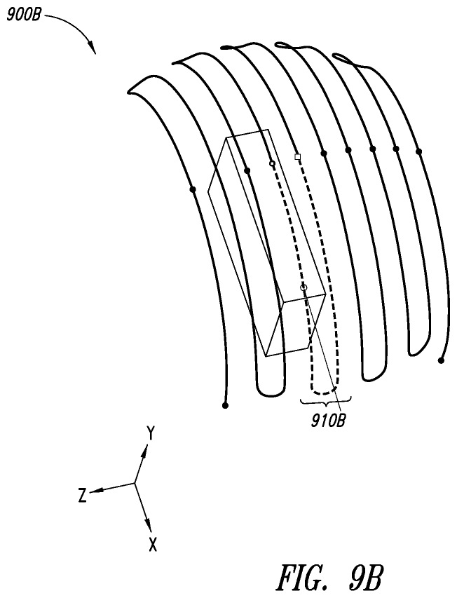

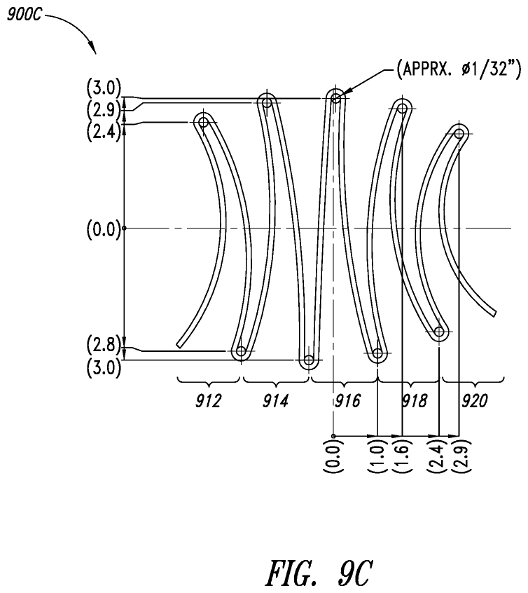

Any of the sensor assemblies disclosed herein can include an antenna configured to transmit and receive in the MICS band, ISM band, Bluetooth band, or another unlicensed band (such as, 928 MHz). As described herein, designing an antenna for operating in a lower frequency range would increase the range of the antenna, but also increase its size. FIG. 9A illustrates a PIFA antenna 900A in the expanded configuration. The antenna 900A can be compressed for loading into a delivery system and expanded when released from the delivery system (and inserted into the treatment site). The antenna 900A can include a plurality of interconnected sections (or segments) 910A. The segments 910A can be of the same or substantially same size and shape. Such configuration can allow the antenna 900A to be compressed and expanded.

FIG. 9B illustrates a PIFA antenna 900B in the expanded configuration. The antenna 900B can be similar to the antenna 900A (with the exception of being longer in the vertical (Y) dimension). The antenna 900B can include a plurality of interconnected segments 910B. The segments 910B can be of the same or substantially same size and shape. Such configuration allows the antenna 900B to be compressed and expanded. The structure behind the antenna 900B can represent a sensor assembly (such as, the sensor assembly 100).

FIG. 9C illustrates a PIFA antenna 900C in a two-dimensional expanded configuration. The antenna 900B can be similar to the antenna 900A (with the exception of having segments of different size and shape). The antenna 900C includes a plurality of interconnected segments 912, 914, 916, 918, and 920. The segments can be of different size and shape to facilitate compression and expansion of the antenna 900C. Such configuration can allow at least some of the segments of the antenna 900C to overlap (or collapse on one another) when the antenna is in the compressed configuration. As a result, the antenna 900C can be smaller in the compressed configuration.

FIG. 10A illustrates a sensor assembly 1000 (which can he similar to the sensor assembly 200) implanted into a treatment site (such as, the aneurysm 520). The sensor assembly 1000 includes an antenna 1012. The antenna 1012 can be a PIFA antenna (such as, the antenna 712). The antenna 1012 can overlie a portion of an anchoring structure 1006 (such as, a coil structure). For example, the antenna 1012 can overlie the top portion of the anchoring structure 1006 and be positioned at the tip of the aneurysm 520. The anchoring structure 1006 can support the antenna 1012 and vice versa.

In some implementations, a plurality of antennas can be used. The antennas can be configured to operate on different frequency bands. In some cases, one of the antennas can be configured to facilitate wireless charging (such as, a coil antenna for inductive charging). With reference to FIGS. 10B and 10C, the sensor assembly 1000 can include two antennas 1012A and 1012B, which can be PIFA antennas. The antennas 1012A and 1012B can be positioned on opposite sides of an anchoring structure (such as, the anchoring structure 1006). For instance, the antennas 1012A and 1012B can overlie opposite sides of the anchoring structure. Advantageously, antenna 1012A can provide structural support for the aneurysm and antenna 1012B can occlude the aneurysm.

FIG. 10D illustrates an antenna 1012D implanted into a treatment site (such as, the aneurysm 520). The antenna 1012D can be a PIFA antenna (such as, the antenna 712). FIG. 10E illustrates another view of the antenna 1012D inside the aneurysm 520. As is illustrated in FIGS. 10D and 10E, the antenna 1012D contours to the top interior portion of the aneurysm 520. FIG. 10F illustrates a side view of the antenna 1012D. FIG. 10G illustrates a top view of the antenna 1012D. FIG. 10H illustrates a perspective view of the antenna 1012D.

Any of the antenna configurations disclosed herein can be integrated with sensor assemblies configured to be implanted not only into the cranium, but also into the lungs, chest, thoracic region, vascular and venous arteries, among others. For example, any of the antenna configurations can be integrated with a sensor assembly configured to be implanted in the bronchioles for monitoring chronic obstructive pulmonary disease (COPD).

### B. Sensor

With reference to FIGS. 2A to 2D and 3, the sensor assembly 100 (or 200 or any of the other sensor assemblies disclosed herein) can include one or more sensors 102. “Sensor” refers to a device that can be utilized to do one or more of detect, measure and/or monitor: one or more different aspects of a body tissue (e.g., anatomy, physiology, metabolism, and function); one or more aspects of body or body segment condition or function (e.g., clotting in an aneurysm); and/or one or more aspects of the sensor assembly 100.

Representative examples of sensors suitable for use within the sensor assembly 100 include, for example, fluid pressure sensors, fluid volume sensors, contact sensors, position sensors, pulse pressure sensors, blood volume sensors, blood flow sensors, chemistry sensors (e.g., for blood and/or other fluids), metabolic sensors (e.g., for blood and/or other fluids), impedance sensors, electrodes, accelerometers, gyroscopes, mechanical stress sensors and temperature sensors. Within some configurations, at least one sensor of the one or more sensors 102 can have a Unique Sensor Identification number (“USI”), which specifically identifies the sensor.

The one or more sensors 102 may be configured to detect, measure and/or monitor information relevant to the state of the sensor assembly 100 after implantation. The state of the sensor assembly 100 may include the integrity of the sensor assembly 100, the movement of the sensor assembly 100, the forces exerted on the sensor assembly 100 and other information relevant to the implanted sensor assembly 100.

The one or more sensors 102 may be configured to detect, measure and/or monitor information relevant body tissue (e.g., one or more physiological parameters of a patient) after implantation of the sensor assembly 100. Body tissue monitoring may include blood pressure, pH level, oxygen, carbon dioxide, potassium, iron, and/or glucose in the blood of the patient. The one or more sensors 102 can include fluid pressure sensors, fluid volume sensors, pulse pressure sensors, blood volume sensors, blood flow sensors, chemistry sensors (e.g., for blood and/or other fluids), metabolic sensors (e.g., for blood and/or other fluids).

A radiopaque marker, or other type of marker, can be integrated with the one or more sensors 102. The radiopaque marker can track a location of the one or more sensors 102 within the vasculature with standard fluoroscopy techniques.

One or more sensors 102 can include a sensing mechanism based on a chemical reaction CR. For example, the sensor(s) 102 can include an outer membrane including a specific stoichiometry and analyte perfusion rate sufficient to manage the chemical reaction CR on the sensor(s) 102 and the resultant output interaction on a platinum surface, such as the antenna(s) 112, that generates a signal for transmission and monitoring of the zone. The signal can determine the chemical reaction CR is in a mode of action of decreasing action or increasing action of the biological transmission of blood from the parent artery to the aneurysm, a void, through a broken containment method for used on addressing an aneurysm closing treatment, cancer, an embolic treatment, an embolic vessel closing treatment (artery or vein), or the like.

The duration of the chemical reaction CR can be greater than or equal to one day and/or less than or equal to 360 days. For example, the duration can be greater than or equal to one day and/or less than or equal to 180 days. The duration can be greater than or equal to one day and/or less than or equal to about 90 days. The duration can be greater than or equal to one day and/or less than or equal to about 30 days. The duration can be greater than or equal to one day and/or less than or equal to about 10 days. The data output variability of the chemical reaction CR can range from a small deviation of 1% to a significant deviation or greater than 99% based on a measurement indication of whether there is a chemical reaction CR or no chemical reaction CR. For example, a conductivity switch or sensor can be used, which is further described below. Based on the chemical interaction, the sensor(s) 102 can detect the resulting biological reaction, which can be used to determine a measurement of biological flow reduction/restriction, biological flow impaction and/or biologic seal. The degradation and/or non-degradation of the signal can be a determination of a function of a treatment in the area/zone in which the sensor assembly 100 and/or the sensor system 150 is placed.

### C. Processor/Controller

The sensor assembly 100 (or 200 or any of the other sensor assemblies disclosed herein) may include a processor (or controller) in electrical communication with the one or more sensors 102 and/or the antenna(s) 112. The one or more sensors 102 and the processor can be located on a printed circuit board. Alternatively, some or all of the one or more sensors 102 may be located in or on another structure of the sensor assembly 100 separate from the printed circuit board. The processor, which can be any suitable microcontroller or microprocessor, can be configured to control the configuration and operation of one or more of the other components of the sensor assembly 100. For example, the processor can be configured to control the one or more sensors 102 to sense relevant measurement data or physiological parameters, to store the measurement data generated by the one or more sensors in a memory, to generate messages, include the stored data as a payload, to packetize the messages, to provide the message packets to the antenna(s) 112 for transmission to a receiver (e.g., hub in the patient's body or a base station or other computing device outside the patient's body). The processor can be configured to execute commands received from a base station or other computing device via the antenna(s) 112. For example, the processor can be configured to receive configuration data from the base station, and to provide the configuration data to the component of the sensor assembly 100 to which the base station directed the configuration data. If the base station directed the configuration data to the processor, then the processor can configure itself in response to the configuration data.

The processor can cause the one or more sensors 102 to measure, to detect, to determine if a measurement is a qualified or valid measurement, to store the data representative of a valid measurement, and to cause the antenna(s) 112 to transmit the stored data to a base station or other source external to the sensor assembly 100. In response to being polled by a base station or by another device external to the sensor assembly 100, the processor can generate conventional messages having payloads and headers. The payload scan can include the stored samples of the signals that the one or more sensors 102 generated. The headers can include the sample partitions in the payload, a time stamp indicating the time at which the sensor 102 acquired the samples, an identifier (e.g., serial number) of the sensor assembly 100, and/or a patient identifier (e.g., a number or name).

The processor can generate data packets that include the messages according to a conventional data-packetizing protocol. Each packet can also include a packet header that includes, for example, a sequence number of the packet so that the receiving device can order the packets properly even if the packets are transmitted or received out of order. The processor can encrypt some or all parts of each of the data packets, for example, according to a conventional encryption algorithm, and error encodes the encrypted data packets. For example, the processor can encrypt at least the sensor assembly 100 and patient identifiers to render the data packets compliant with the Health Insurance Portability and Accountability Act (“HIPAA”). The processor can provide the encrypted and error-encoded data packets to the antenna(s) 112, which, via the filter, transmits the data packets to a destination, such as the base station 4 (shown in FIG. 1) or a receiver external to the sensor system 150. The antenna(s) 112 can transmit the data packets according to any suitable data-packet-transmission protocol.

Alternate configurations of the sensor assembly 100 and/or the sensor system 150 are contemplated. For example, the antenna(s) 112 can perform encryption or error encoding instead of, or complementary to, the processor. Furthermore, the sensor assembly 100 and the sensor system 150 can include components other than those described herein and can omit one or more of the components described herein.

The sensor assembly 100 may include a memory circuit (not shown) that can be any suitable nonvolatile memory circuit, such as EEPROM or FLASH memory. The memory can be in electrical communication with the processor, the antenna(s) 112, and/or the one or more sensors 102. The memory can be configured to store data written, for example, by the processor or the antenna(s) 112, and to provide data in response to a read command from the processor.

### D. Power Source

The sensor assembly 100 (or 200 or any of the other sensor assemblies disclosed herein) can include one or more power sources. For example, the sensor assembly can include one or more batteries and/or supercapacitors. The power source may be sized to fit within the vascular structure with the remainder of the sensor assembly 100. In other configurations, the sensor assembly 100 may be powered by a power source at a remote location from the vascular structure, either in the patient's body or outside the patient's body.

The power source can be any suitable battery, such as a Lithium Carbon Monofluoride (LiCFx) battery or solid state battery, or other storage cell capable of storing energy (e.g., a supercapacitor) for powering the processor for an expected lifetime of the sensor assembly 100 (e.g., at least one month or at least six months). The power source may receive sufficient energy from the sensor reaction by-product to maintain a minimal power capacity for sustaining micro-controller memories, real time clocks and/or SRAMs sleep modes.

Replacing a power source implanted in a patient is often desirable at least because it involves an invasive procedure that can be relatively expensive and that can have adverse side effects, such as infection and soreness. Thus, the power source may be rechargeable. For example, the power source may be recharged using integrated circuitry on an ASIC chip. As another example, the battery may be charged inductively. For example, any of the sensor assemblies disclosed herein can include an antenna for wireless charging (such as, a coil antenna). Any of garments disclosed herein can include a wireless power transmitter to facilitate inductive charging.

### III. METHODS OF USE OF THE SENSOR SYSTEM

Any of the implantable sensor assemblies 100 (or 200 or any of the other sensor assemblies disclosed herein) and/or the implantable sensor systems 150 described herein can be implanted into a patient to monitor any anatomical structure of the patient. For example, the sensor assemblies 100 and/or the implantable sensor systems 150 can be implanted into an aneurysm for monitoring blood flow into the aneurysm. Less blood flow can indicate that the aneurysm is clotting, while more blood flow can indicate that the aneurysm is not clotting.

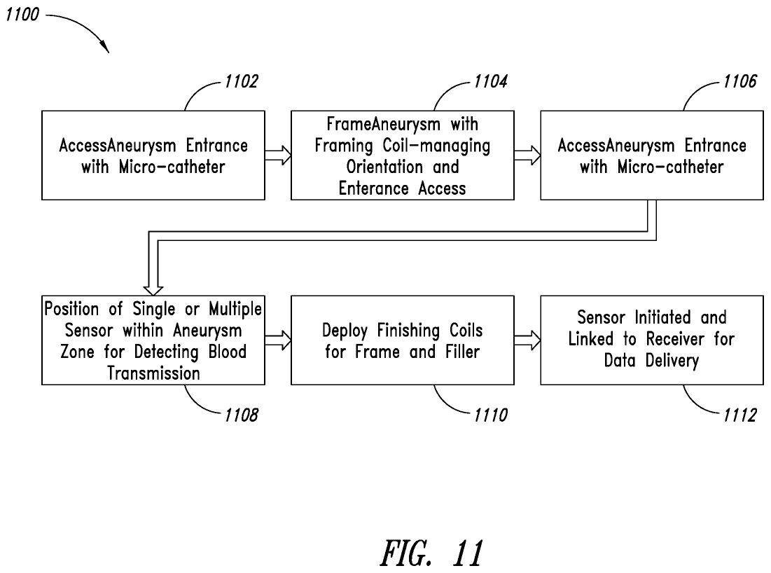

The implantable sensor assembly 100 (or 200 or any of the sensor assemblies disclosed herein) and/or the implantable sensor system 150 can be implanted into the patient via a delivery system. FIG. 11 illustrates an example method 1100 of delivering the sensor assembly 100 and/or the sensor system 150 into the anatomical structure of interest (e.g., an aneurysm). Although the method 1100 is described with respect to an aneurysm, the method may be used to deliver the sensor assembly 100 to other vascular structures. Using imaging techniques, the clinician can identify the anatomical structure of interest in the patient's body. At block 1102, the clinician can advance a guide structure, such as a guidewire or guide catheter, to the target site. Optionally, at block 1104, the clinician can frame the aneurysm with one or more framing coils for managing the orientation and aneurysm access. At block 1106, the clinician can position a distal end of the delivery system through the aneurysm neck. For example, the clinician can position the distal end of the delivery system through approximately the middle of the neck. At block 1108, the clinician can position a sensor assembly 100 within the aneurysm, for example at or near the middle of the aneurysm or away from the aneurysm walls and neck, such that the sensor assembly 100 can detect blood flow into the aneurysm. At block 1110, the clinician can deploy an anchoring structure 106 (e.g., a metal coil or basket) around the sensor assembly 100 or between the sensor assembly 100 and a wall of the vascular structure. At block 1112, once the sensor system 150 is implanted in the aneurysm, the clinician can initiate the sensor system 150 and link it to a receiver to receive sensor data from the sensor system 150.

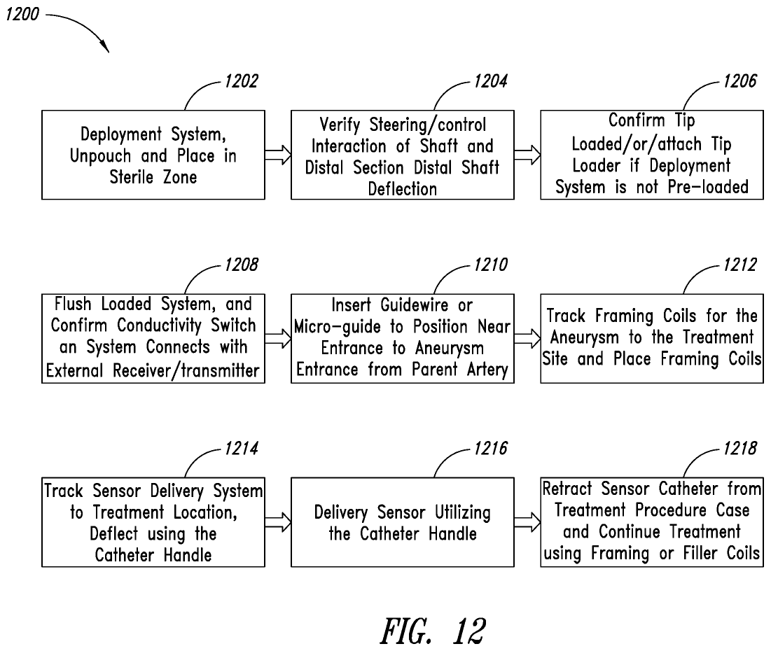

FIG. 12 illustrates another example method 1200 for delivering the sensor assembly 100 (or 200 or any of the other sensor assemblies disclosed herein) and/or sensor system 150 to a patient's anatomical structure of interest, for example an aneurysm. Although the method 1200 is described with respect to an aneurysm, the method may be used to deliver the sensor assembly 100 to other vascular structures. At block 1202, the delivery or deployment system can be unpacked and placed in a sterile location. At block 1204, the clinician can verify the steering and control of the shaft of the delivery system, and the deflection of the distal section of the shaft. At block 1206, the clinician can confirm the distal tip of the delivery system is loaded with the sensor assembly 100. If the delivery system is not pre-loaded with the sensor assembly 100, the clinician can load the distal tip with the sensor assembly 100. At block 1208, the medical practitioner can flush the loaded delivery system and confirm that the conductivity switch of the sensor assembly 100, if present, is functioning and that the sensor assembly 100 is capable of connecting with an external transceiver. At block 1210, the medical practitioner can insert a guidewire or guide catheter to a position near the entrance or neck of the aneurysm. Optionally, at block 1212, the clinician can deliver one or more framing coils to the aneurysm. At block 1214, the clinician can deliver the delivery system to the aneurysm by using the catheter handle to deflect the distal section of the shaft as needed. At block 1216, the medical practitioner can deliver the sensor assembly 100 into the aneurysm. Once the sensor assembly 100 is implanted in the aneurysm, at block 1218, the clinician can retract the catheter from the patient and implant an anchoring structure 106, such as framing or filler coils, around the sensor assembly 100 or between the sensor assembly 100 and a wall of the vascular structure. Once the sensor system 150 is implanted in the aneurysm, the clinician can initiate the sensor assembly 150 and link it to a receiver to receive sensor data from the sensor system 150.

Once the sensor system 150 is implanted into the patient and initially configured, the sensor system 150 can begin generating continuously or intermittently sensor data related to one or more physiological parameters of the patient. For example, when a conductivity sensor switch of the sensor system 150 is exposed to the patient's blood, the sensor system 150 can switch on begin detecting one or more physiological parameters of the patient. The sensor system 150 can transmit the sensor data to a receiver continuously, intermittently at a regular or irregular time interval, or upon command. The receiver can be, for example, a base station, a smart device, a computing device or an external transceiver.

### IV. KIT

Any of the implantable sensor assemblies and/or anchor structures described herein may be provided in a kit with one or more delivery systems. The same delivery system may be used to deliver the sensor assembly and the anchor structure. Alternatively, the kit may include separate delivery systems for the sensor assembly and the anchor structure.

The delivery system may be pre-loaded with the sensor assembly prior to packaging or provided in the kit separate from the sensor assembly. When separately provided, the delivery system may be loaded with the sensor assembly by the clinician.

The delivery system may include a loading chamber for carrying the sensor assembly. For example, the loading chamber may be provided in a lumen of the delivery system. The lumen may carry a release mechanism, such as a pusher, to release the sensor system from the delivery system. The loading chamber may be positioned in the same lumen or a different lumen as the guidewire. The loading chamber may be separate and distinct from the guidewire lumen and/or fluid delivery lumen.

The delivery system may include a deflectable distal tip. The deflectable tip may include a radiopaque marker to track a location of the delivery system within the vasculature with standard fluoroscopy techniques. The deflectable distal tip may be actively and/or passively deflected to steer the sensor assembly to the target site. In delivery systems with active deflection, the delivery system may include a handle capable of mechanically and/or electrically steering the deflectable distal tip. For example, the handle may directly cause deflection of the deflectable distal tip through one or more cables, wires, or other connection between the handle and the deflectable distal tip. As another example, the handle may indirectly cause deflection of the deflectable distal tip by deflecting an outer sleeve, which forces deflection of the deflectable distal tip.

The delivery system may be provided with an adaptor for connection to a robotic surgical system. The clinician may use the robotic surgical system to actively steer the delivery system to the target site. Robotic surgical systems, teleoperated surgical systems, and the like, which may be used or adapted to connect with a delivery system of the present disclosure so as to deliver and implant an implantable sensing assembly of the present disclosure into a patient, have been commercialized by several companies. One example of such a teleoperated, computer-assisted surgical system (e.g., a robotic system that provides telepresence) with which embodiments of the present disclosure may be used, are the da Vinci Surgical Systems manufactured by Intuitive Surgical, Inc. of Sunnyvale, Calif, USA. See, e.g., U.S. Pat. Nos. 9,358,074; 9,295,524; and 8,852,208; U.S. Patent Publication Nos. 20140128886; 20200253678; 20190192132; 20190254763; 20180318020; 20170312047; 20170172671; 20170172674; 20170000575; 20170172670; 20130204271; and 20120209305; and PCT Publication No. WO2020150165, each of which is incorporated by reference. Another example is Medtronic, Inc. (Minneapolis, MN, USA; and related companies, e.g., Covidien LP, Mansfield MA USA and Medtronic Navigation, Inc., Louisville CO USA) including their Digital Surgery Division and Surgical Robotics Division, which has commercialized various robotic-assisted surgery (RAS) solutions. See, e.g., U.S. Patent Publication Nos. 20200222127; 20190365477; 20190214126; 20190069964; and 20130289439, each of which is incorporated by reference. Yet another example is Auris Health (Redwood City, CA USA; Auris Health, Inc., is part of Johnson & Johnson Medical Devices Companies. Auris Health, Inc. was formerly known as Auris Surgical Robotics, Inc.) which has commercialized their Monarch platform. See, e.g., U.S. Patent Publication Nos. 20200198147; 20200100845; 20200100853; 20200100855; 20200093554; 20200060516; 20200046434; 20200000537; 20190365209; and 20190365486, each of which is incorporated by reference. In addition, Stryker Corp. (Kalamazoo MI USA) discloses robotic surgical systems in, e.g., U.S. Patent Publication Nos. 20160374770 and 20140276949, both of which are incorporated by reference. See also, e.g., U.S. Patent Publication Nos. 20200046978; 20200001053; 20200197111; 20190262084; 20190231447; and 20190090957 and PCT Publication Nos. WO2019195841 and WO2019082224, where each of the identified publications is incorporated by reference. In one embodiment, the handle of the delivery system of the present disclosure is configured to dock with an arm of a robotic surgical system. In one embodiment, the delivery system of the present disclosure integrates with a robotic surgical system to provide robot-assisted delivery and implantation of the implantable sensing assembly of the present disclosure into a patient. In one embodiment, the present disclosure provides a method for advancing any of the implantable sensor assemblies and/or systems described herein through the vasculature of a patient, using robotic assistance.

### V. DELIVERY SYSTEM

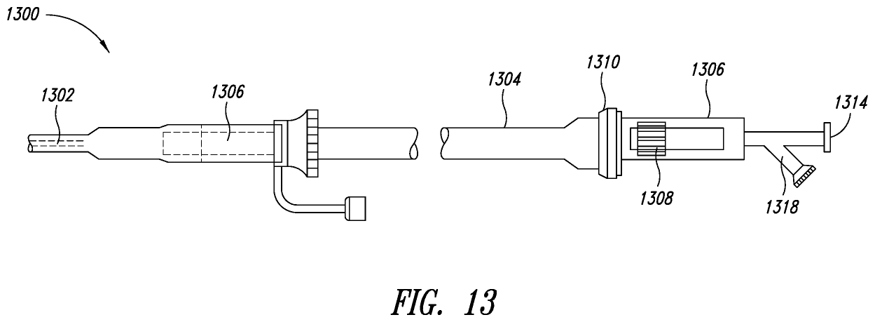

FIG. 13 illustrates a delivery system 1300 for advancing any of the implantable sensor assemblies, which can include one or more antennas, and/or systems, which can include one or more antennas, described herein through the vasculature. The delivery system 1300 may include a deflectable distal tip 1302, a handle 1306, and a shaft 1304 therebetween. In some contexts, the delivery system 1300 may be sufficiently sized to be advanced to the neuro-vasculature. For example, an outer diameter of the shaft 1304 may be less than or equal to 1 mm. As used herein, the relative terms “proximal” and “distal” shall be defined from the perspective of the delivery system. Thus, proximal refers to the direction of the control end of the delivery system and distal refers to the direction of the distal tip.

The deflectable distal tip 1302 may carry the sensor assembly. For example, the deflectable distal tip 1302 may be pre-loaded with the sensor assembly prior to introducing the sensor assembly into the patient. The sensor assembly may be pre-loaded in a loading chamber separate from a guidewire lumen or fluid delivery lumen. The sensor assembly may be attached to the loading chamber or freely sit within the loading chamber. The sensor assembly may be sterilized prior to loading or sterilized together with the delivery system 1300. In other delivery methods, the sensor assembly may be advanced to the deflectable distal tip 1302 after the delivery system 1300 has been advanced to the target site. One or more antennas of the sensor assembly can be in the compressed configuration when the sensor assembly is loaded into and carried by the delivery system 1300.

The deflectable tip 1302 may be actively deflected using the handle 1306 to facilitate accurate placement of the sensor assembly. For example, the deflectable tip 1302 may be mechanically deflected using a user-actuatable mechanism in the handle 1306. The user-actuatable mechanism may control one or more cables or wires extending through the wall of the shaft 1304 or along an inner and/or outer surface of the shaft 1304 to manipulate the deflectable distal tip 1302. Additionally or alternatively, the deflectable distal tip 1302 may be sufficiently flexible to be passively deflected. The deflectable distal tip 1302 may include one or more markers to monitor a position and/or direction of the deflectable distal tip.

The deflectable distal tip 1302 may be constructed of one or more polymeric materials, such as Pebax® polyethylene, polyethylene terephthalate, or other polymeric materials. The deflectable distal tip 1302 may or may not be supported by a braided material.

The shaft 1304 may include one or more internal lumens. For example, the shaft 1304 may have a guidewire lumen for tracking the delivery system 1300 to the target site. The guidewire lumen may extend from the guidewire lumen port 1314 in the handle 1306 and through the deflectable distal tip 1302. The shaft 1304 may have a fluid delivery lumen to delivery fluid to the delivery site.

The shaft 1304 may be constructed of one or more polymeric materials, such as Pebax® polyethylene, tetrafluoroetheylene, polytetrafluoroethylene, or other polymeric materials. The shaft 1304 may be reinforced with a braided material to enhance pushability and/or torque management. The shaft 1304 may be co-extruded with a first polymeric material and a liner and/or outer layer. The liner and/or outer layer may include tetrafluoroetheylene or polytetrafluoroethylene.

The handle 1306 may include one or more user-actuatable mechanisms for controlling different functions of the delivery system 1300. For example, the handle 1306 may include a first user-actuatable mechanism 1310 capable of releasing the sensor assembly from the delivery system 1300. The handle 1306 may include a second user-actuatable mechanism 1308 capable of steering the shaft 1304 and/or the deflectable distal tip 1302. As used herein, the terms “first” and “second” user-actuatable mechanism can be used interchangeably. For example, the “first” user-actuatable mechanism may refer to any control feature described herein.

The first user-actuatable mechanism 1310 may push the sensor assembly out of the distal tip 1302 of the delivery system 1300. For example, the first user-actuatable mechanism 1310 may control a pusher extending through a lumen in the shaft 1304. In another configuration, the first user-actuatable mechanism 1310 may withdraw the distal tip 1302 relative to the sensor assembly to release the sensor assembly. As illustrated, the first user-actuatable mechanism 1310 may be an axial slider, but in other configurations, the first user-actuatable mechanism 1310 may be a button, switch, lever, rotatable knob, rotatable dial, or otherwise. One or more antennas of the sensor assembly can transition from the compressed configuration into the expanded configuration as the sensor assembly is being released (such as, pushed out of the distal tip 1302) or subsequently to the sensor assembly being released (such as, subsequently to being pushed out of the distal tip 1302).

The second user-actuatable mechanism 1308 may steer the shaft 1304 and/or the deflectable distal tip 1302. As illustrated, the second user-actuatable mechanism 1308 may be a rotary knob capable of controlling a direction of the flexible shaft 1304 and/or the distal tip 1302. The rotary knob may rotate about a longitudinal axis of the handle 1306. In other configurations, the second user-actuatable mechanism 1310 may rotate in a different direction.

As shown in FIG. 13, the first user-actuatable mechanism 1310 may be positioned proximally of the second user-actuatable mechanism 1308. In other configurations, the second user-actuatable mechanism 1308 may be positioned proximally or elsewhere relative to the first user-actuatable mechanism 1310.

The handle 1306 may also include one or more ports. For example, the handle 1306 may include a flush port 1312 for introducing fluid into the delivery system 1300. The handle 1306 may include a separate guidewire lumen port 1314. As illustrated, the one or more ports are positioned proximally of the user-actuatable mechanisms, but may be positioned anywhere along the delivery system 1302.

The handle 1306 may be molded from a polymeric material. For example, the polymeric material may include ABS, polypropylene, Pebax®, or other materials.

Optionally, the delivery system 1300 may include a delivery sheath 1316 positioned over the shaft 1304. The delivery sheath 1316 may act as an introducer. The delivery sheath 1316 may include one or more seals to prevent fluid flow out of the patient from a space between the delivery sheath 1316 and the shaft 1304. For example, the delivery sheath 1316 may include a seal near a proximal end of the delivery sheath 1316. The delivery sheath 1316 may include a separate port 1318 to flush the delivery sheath 1316 or lubricate the interaction between the delivery sheath 1316 and the shaft 1304.

In some configurations, the delivery sheath 1316 may enhance steerability and trackability of the delivery system 1300 through the vasculature. For example, the delivery sheath 1316 may be connected to the deflectable distal tip 1302 to enable steering of the deflectable distal tip 1302. As another example, the delivery sheath 1316 may not engage the deflectable distal tip 1302, but bending of the delivery sheath 1316 forces deflection of the distal tip 1302.

The delivery sheath 1316 may be constructed of a same or different material as the deflectable distal tip 1302. For example, the delivery sheath 1316 may be constructed of a polymeric material, such as Pebax® polyethylene, polyethylene terephthalate, or other polymeric materials. The delivery sheath 1316 may or may not be supported by a braided material.

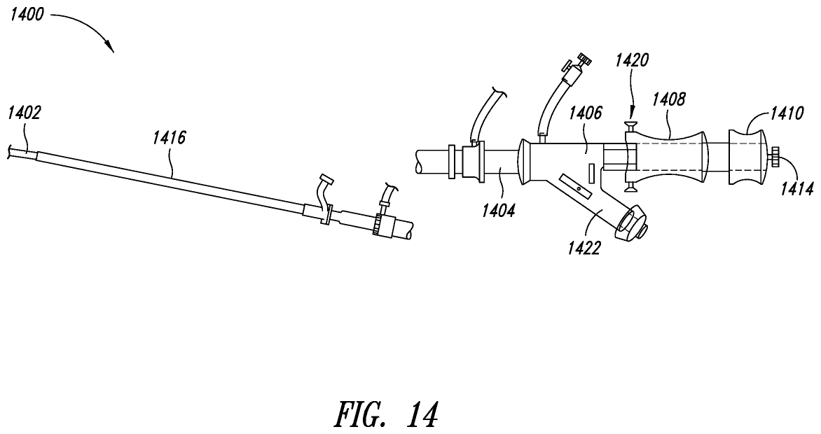

FIG. 14 illustrates another delivery system 1400 for advancing any of the implantable sensor assemblies, which can include one or more antennas, or systems, which can include one or more antennas, described herein through the vasculature. The delivery system 1400 may include any of the features described above with respect to the delivery system 1400. The delivery system 1300 may include a deflectable distal tip 1402, a handle 1406, and a shaft 1404 therebetween.

The deflectable distal tip 1402 may include a loading chamber for carrying the sensor assembly. The loading chamber may be separate from any guidewire, fluid delivery lumen, and/or other lumen extending through the deflectable distal tip 1402. One or more antennas of the sensor assembly can be in the compressed configuration when the sensor assembly is loaded into and carried by the delivery system 1400.

The deflectable distal tip 1402 may include a molded or thermally reshaped polymer. For example, the deflectable distal tip 1402 may include one or more polymeric materials, such as Pebax® polyethylene, tetrafluoroetheylene, polytetrafluoroethylene, or other polymeric materials. The deflectable distal tip 1402 may or may not be supported by a braided material. For example, the deflectable distal tip 1402 may include a braided structure lined with and/or coated or over-molded with a separate polymeric layer such as tetrafluoroetheylene or polytetrafluoroethylene.

The shaft 1404 may include one or more internal lumens. For example, the shaft 804 may have a guidewire lumen for tracking the delivery system 1400 to the target site. The guidewire lumen may extend from the guidewire lumen port 1414 in the handle 1406 and through the deflectable distal tip 1402. The shaft 1404 may have a fluid delivery lumen to delivery fluid to the delivery site. The shaft 1404 may include one or more polymeric materials, such as Pebax® polyethylene, tetrafluoroetheylene, polytetrafluoroethylene, or other polymeric materials.

The handle 1406 may include one or more user-actuatable mechanisms for controlling different functions of the delivery system 1400. For example, the handle 1406 may include a first user-actuatable mechanism 1410 capable of releasing the sensor assembly from the delivery system 1400. One or more antennas of the sensor assembly can transition from the compressed configuration into the expanded configuration as the sensor assembly is being released or subsequently to the sensor assembly being released. The handle 1406 may include a second user-actuatable mechanism 1408 capable of steering the shaft 1404 and/or the deflectable distal tip 1402. The second user-actuatable mechanism 1408 may be rotatable knob, but in other configurations, the second user-actuatable mechanism 1408 may be a button, switch, lever, slider, rotatable dial, or otherwise. The handle 1406 may include a third user actuatable mechanism 1420 to stabilize the shaft 1404 and/or the orientation of the deflectable distal tip 1402. For example, the third user actuatable mechanism 1420 may be a toggle lock. As used herein, the terms “first,” “second” and “third” user-actuatable mechanism can be used interchangeably. For example, the “first” user-actuatable mechanism may refer to any control feature described herein. The user-actuatable mechanisms may be positioned in an order corresponding to their usage during a procedure.

The handle 1406 may include a control 1422 for steering the deflectable tip 1402 and/or the shaft 1404. For example, the handle 1406 may include a mechanical and/or electrical control mechanism for steering the deflectable distal tip 1402 and/or the shaft 1404. For example, the delivery system 1400 can include a slider or carriage assembly with one or more wires or cables. The delivery system 1400 can include a voltage control to activate the deflection mechanism. The wires or cables can electrically transmit the energy to steer the deflectable distal tip 1402 and/or shaft 1404. Additionally or alternatively, this mechanical and/or electrical control mechanism may be applied to a delivery sheath positioned over the shaft 1404.

### VI. ADDITIONAL EMBODIMENTS AND TERMINOLOGY

All of the U.S. patents, U.S. patent application publications, U.S. patent applications, foreign patents, foreign patent applications and non-patent publications referred to in this specification and/or listed in the Application Data Sheet, are incorporated herein by reference, in their entirety. Such documents may be incorporated by reference for the purpose of describing and disclosing, for example, materials and methodologies described in the publications, which might be used in connection with the presently described invention. The publications discussed above and throughout the text are provided solely for their disclosure prior to the filing date of the present application. Nothing herein is to be construed as an admission that the inventors are not entitled to antedate any referenced publication by virtue of prior invention.

Although certain methods have been described herein with respect to aneurysms, the methods described herein can be applied to any vascular structure, for example a ductus arteriosus Although certain embodiments and examples have been described herein, it will be understood by those skilled in the art that many aspects of the sensor assemblies shown and described in the present disclosure may be differently combined and/or modified to form still further embodiments or acceptable examples. All such modifications and variations are intended to be included herein within the scope of this disclosure. A wide variety of designs and approaches are possible. No feature, structure, or step disclosed herein is essential or indispensable.

For purposes of this disclosure, certain aspects, advantages, and novel features are described herein. It is to be understood that not necessarily all such advantages may be achieved in accordance with any particular embodiment. Thus, for example, those skilled in the art will recognize that the disclosure may be embodied or carried out in a manner that achieves one advantage or a group of advantages as taught herein without necessarily achieving other advantages as may be taught or suggested herein.

Moreover, while illustrative embodiments have been described herein, the scope of any and all embodiments having equivalent elements, modifications, omissions, combinations (e.g., of aspects across various embodiments), adaptations and/or alterations as would be appreciated by those in the art based on the present disclosure. The limitations in the claims are to be interpreted broadly based on the language employed in the claims and not limited to the examples described in the present specification or during the prosecution of the application, which examples are to be construed as non-exclusive. Further, the actions of the disclosed processes and methods may be modified in any manner, including by reordering actions and/or inserting additional actions and/or deleting actions. It is intended, therefore, that the specification and examples be considered as illustrative only, with a true scope and spirit being indicated by the claims and their full scope of equivalents.

The terms “approximately,” “about,” and “substantially” as used herein represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

Conditional language used herein, such as, among others, “can,” “might,” “may,” “e.g.,” and the like, unless specifically stated otherwise, or otherwise understood within the context as used, is generally intended to convey that some embodiments include, while other embodiments do not include, certain features, elements, and/or states. Thus, such conditional language is not generally intended to imply that features, elements, blocks, and/or states are in any way required for one or more embodiments or that one or more embodiments necessarily include logic for deciding, with or without author input or prompting, whether these features, elements and/or states are included or are to be performed in any particular embodiment.

The methods disclosed herein may include certain actions taken by a clinician; however, the methods can also include any third-party instruction of those actions, either expressly or by implication. For example, actions such as “releasing the sensor assembly” include “instructing release of the sensor assembly.”

The various illustrative logical blocks, modules, routines, and algorithm steps described in connection with the embodiments disclosed herein can be implemented as electronic hardware, computer software, or combinations of both. To clearly illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a general purpose processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the signal processing algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An example storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

### VII. EXAMPLE EMBODIMENTS

The following example embodiments identify some possible permutations of combinations of features disclosed herein, although other permutations of combinations of features are also possible.
1. An implantable system for treating a cranial aneurysm, the system comprising:
   a plurality of coils configured to be positioned within the cranial aneurysm to cause an occlusion of a flow of blood in the cranial aneurysm;
   a sensor configured to be positioned within the cranial aneurysm and further configured to detect a property of the flow of blood;
   a communication circuitry configured to be positioned within the cranial aneurysm, the communication circuitry connected to the sensor and configured to transmit data related to the property of the flow of blood detected by the sensor; and
   an antenna configured to be positioned within the cranial aneurysm, the antenna connected to the communication circuitry and further configured to facilitate transmission of the data related to the property of the flow of blood detected by the sensor.
2. The implantable system of Embodiment 1, wherein the plurality of coils, the sensor, the communication circuitry, and the antenna are configured to be loaded into a catheter and inserted into the cranial aneurysm using the catheter.
3. The implantable system of Embodiment 2, wherein the antenna is configured to compress into a first shape to be loaded into the catheter, and wherein the antenna is configured to expand into a second shape subsequent to being released from the catheter.
4. The implantable system of Embodiment 3, wherein the antenna comprises a plurality of interconnected segments.
5. The implantable system of Embodiment 3 or 4, wherein the antenna in the second shape forms a substantially semi-spherical structure that facilitates occlusion of the flow of blood.
6. The implantable system of Embodiment 3 to 5, wherein the antenna comprises first and second materials, the first material configured to facilitate compression of the antenna into the first shape and expansion of the antenna into the second shape, and the second material configured to facilitate the transmission.
7. The implantable system of Embodiment 6, wherein the first material comprises a shape memory alloy, and the second material comprises one or more of platinum, platinum and iridium alloy, or gold.
8. The implantable system of any one of the preceding Embodiments, wherein the antenna comprises a flat monopole antenna.
9. The implantable system of any one of the preceding Embodiments, wherein the antenna comprises a planar inverted-F antenna (PIFA).
10. The implantable system of any one of the preceding Embodiments, wherein the antenna is connected to the plurality of coils such that the plurality of coils serves as a ground plane for the antenna.
11. The implantable system of Embodiment 10, wherein the antenna is connected to the plurality of coils through a single electrical connection.
12. The implantable system of Embodiment 10, wherein the antenna is connected to the plurality of coils through a plurality of electrical connections.
13. The implantable system of any one of the preceding Embodiments, wherein, subsequent to being inserted into the cranial aneurysm, the antenna is positioned outside of a structure formed by the plurality of coils, and wherein the sensor and the communication circuitry are positioned at least partially inside the structure formed by the plurality of coils.
14. The implantable system of Embodiment 13, wherein the plurality of coils are configured to form a substantially spherical shape when positioned within the cranial aneurysm.
15. The implantable system of any one of the preceding Embodiments, wherein the antenna, in an expanded configuration, is configured to form a substantially semi-spherical structure that facilitates occlusion of the flow of blood.
16. The implantable system of any one of the preceding Embodiments, wherein the antenna comprises first and second antennas configured to communicate in different frequency bands.
17. The implantable system of Embodiment 16, wherein the first antenna, in an expanded configuration, is configured to form a first substantially semi-spherical structure that contours to one or more walls of the cranial aneurysm, and wherein the second antenna, in an expanded configuration, is configured to form a second substantially semi-spherical structure that facilitates occlusion of the flow of blood.
18. The implantable system of Embodiment 17, wherein the first structure positioned opposite the second structure when the first and second antennas are in the expanded configuration.
19. The implantable system of any one of the preceding Embodiments, wherein the antenna is configured to transmit in a frequency range between 2400 MHz and 2483.5 MHz.
20. The implantable system of any one of the preceding Embodiments, wherein the antenna is configured to transmit and receive data over a distance of at least 12 centimeters.
21. The implantable system of any one of the preceding Embodiments, wherein the sensor comprises at least one of a blood flow sensor, a blood pressure sensor, a metabolic sensor, a glucose sensor, an oxygen sensor, or a pressure sensor.
22. The implantable system of any one of the preceding Embodiments, wherein the antenna comprises or is coated with a material that promotes clotting of blood.
23. The implantable system of any one of the preceding Embodiments, wherein the antenna comprises or is coated with a material that inhibits clotting of blood.
24. The implantable system of any one of the preceding Embodiments, further comprising a receiver configured to receive the data related to the property of blood flow detected by the sensor, wherein the receiver is configured to be supported by an article of clothing worn by a user in the upper torso or head.
25. The implantable system of Embodiment 24, wherein the article of clothing comprises a hat, headband, or scarf.
26. A kit comprising the implantable system of any one of the preceding Embodiments and a delivery system configured to release the implantable system in the cranial aneurysm.
27. An implantable system for monitoring a vascular structure, the system comprising:
   a sensor configured to be positioned within the vascular structure and further configured to detect a property of a flow of blood in the vascular structure;
   a communication circuitry configured to be positioned within the vascular structure, the communication circuitry connected to the sensor and configured to transmit data related to the property of the flow of blood detected by the sensor; and
   an antenna configured to be positioned within the vascular structure, the antenna connected to the communication circuitry and further configured to facilitate transmission of the data related to the property of the flow of blood detected by the sensor.
28. The implantable system of Embodiment 27, wherein the vascular structure comprises a neurovascular structure.
29. The implantable system of any of Embodiments 27 to 28, wherein the vascular structure comprises a cardiovascular structure.
30. The implantable system of any of Embodiments 27 to 29, wherein the vascular structure comprises a respiratory structure.
31. The implantable system of any of Embodiments 27 to 30, wherein the communication circuitry and the antenna are configured to be loaded into a delivery system and inserted into the vascular structure using the delivery system.
32. The implantable system of Embodiment 31, wherein the antenna is configured to compress into a first shape to be loaded into the delivery system, and wherein the antenna is configured to expand into a second shape subsequent to being released from the delivery system.
33. The implantable system of Embodiment 32, wherein the antenna comprises a plurality of interconnected segments.
34. The implantable system of Embodiment 32 or 33, wherein the antenna in the second shape forms a substantially semi-spherical structure that facilitates occlusion of the flow of blood.
35. The implantable system of any of Embodiments 32 to 34, wherein the antenna comprises first and second materials, the first material configured to facilitate compression of the antenna into the first shape and expansion of the antenna into the second shape, and the second material configured to facilitate the transmission.
36. The implantable system of Embodiment 35, wherein the first material comprises a shape memory alloy, and the second material comprises one or more of platinum, platinum and iridium alloy, or gold.
37. The implantable system of any of Embodiments 27 to 36, wherein the antenna comprises a flat monopole antenna.
38. The implantable system of any of Embodiments 27 to 37, wherein the antenna comprises a planar inverted-F antenna (PIFA).
39. The implantable system of any of Embodiments 27 to 38, wherein the antenna is connected to an anchoring structure such that the anchoring structure serves as a ground plane for the antenna.
40. The implantable system of Embodiment 39, wherein the antenna is connected to the anchoring structure through a single electrical connection.
41. The implantable system of Embodiment 39, wherein the antenna is connected to the anchoring structure through a plurality of electrical connections.
42. The implantable system of any of Embodiments 27 to 41, wherein, subsequent to being inserted into the vascular structure, the antenna is configured to be positioned outside of an interior volume of an anchoring structure, and wherein the sensor and the communication circuitry are configured to be at least partially positioned within the interior volume.
43. The implantable system of Embodiment 42, wherein the anchoring structure is configured to form a substantially spherical shape when positioned within the vascular structure.
44. The implantable system of any of Embodiments 27 to 43, wherein the antenna, in an expanded configuration, is configured to form a substantially semi-spherical structure that facilitates occlusion of the flow of blood.
45. The implantable system of any of Embodiments 27 to 44, wherein the antenna comprises first and second antennas configured to communicate in different frequency bands.
46. The implantable system of Embodiment 45, wherein the first antenna, in an expanded configuration, is configured to form a first substantially semi-spherical structure that contours to or more walls of the vascular structure, and wherein the second antenna, in an expanded configuration, is configured to form a second substantially semi-spherical structure that facilitates occlusion of the flow of blood.
47. The implantable system of Embodiment 46, wherein the first structure positioned opposite the second structure when the first and second antennas are in the expanded configuration.
48. The implantable system of any of Embodiments 27 to 47, wherein the antenna is configured to transmit in a frequency range between 2400 MHz and 2483.5 MHz.
49. The implantable system of any of Embodiments 27 to 48, wherein the antenna is configured to transmit and receive data over a distance of at least 12 centimeters.
50. The implantable system of any of Embodiments 27 to 49, wherein the sensor comprises at least one of a blood flow sensor, a blood pressure sensor, a metabolic sensor, a glucose sensor, an oxygen sensor, or a pressure sensor.
51. The implantable system of any of Embodiments 27 to 50, wherein the antenna comprises or is coated with a material that promotes clotting of blood.
52. The implantable system of any of Embodiments 27 to 51, wherein the antenna comprises or is coated with a material that inhibits clotting of blood.
53. The implantable system of any of Embodiments 27 to 52, further comprising a receiver configured to receive the data related to the property of blood flow detected by the sensor, wherein the receiver is configured to be supported by an article of clothing worn by a user in the upper torso or head.
54. The implantable system of Embodiment 53, wherein the article of clothing comprises a hat, headband, or scarf.
55. A kit comprising the implantable system of any of Embodiments 27 to 54 and a delivery system configured to release the implantable system in the vascular structure.
56. A method of implanting, using, or operating the system of any of the preceding Embodiments.
57. The implantable system as described herein, such any of Embodiments 1-25 and 27-54, wherein the antenna comprises platinum and iridium.
58. An antenna as illustrated and/or described.
59. A method of implanting, using, or operating an antenna as illustrated and/or described.
60. An implantable system as illustrated and/or described.
61. A method of implanting, using, or operating an implantable system as illustrated and/or described.

What is claimed is:

1. An implantable system for treating a cranial aneurysm, the system comprising:
a plurality of coils configured to be positioned within the cranial aneurysm to cause an occlusion of a flow of blood in the cranial aneurysm;
a sensor configured to be positioned within the cranial aneurysm and further configured to detect a property of the flow of blood;
a communication circuitry configured to be positioned within the cranial aneurysm, the communication circuitry connected to the sensor and configured to transmit data related to the property of the flow of blood detected by the sensor; and
an antenna configured to be positioned within the cranial aneurysm, the antenna connected to the communication circuitry and further configured to facilitate transmission of the data related to the property of the flow of blood detected by the sensor.

2. The implantable system of claim 1, wherein the plurality of coils, the sensor, the communication circuitry, and the antenna are configured to be loaded into a catheter and inserted into the cranial aneurysm using the catheter.

3. The implantable system of claim 2, wherein the antenna is configured to compress into a first shape to be loaded into the catheter, and wherein the antenna is configured to expand into a second shape subsequent to being released from the catheter.

4. The implantable system of claim 3, wherein the antenna comprises a plurality of interconnected segments.

5. The implantable system of claim 3, wherein the antenna in the second shape forms a substantially semi-spherical structure that facilitates occlusion of the flow of blood.

6. The implantable system of claim 3, wherein the antenna comprises first and second materials, the first material configured to facilitate compression of the antenna into the first shape and expansion of the antenna into the second shape, and the second material configured to facilitate the transmission.

7. The implantable system of claim 6, wherein the first material comprises a shape memory alloy, and the second material comprises one or more of platinum, platinum and iridium alloy, or gold.

8. The implantable system of claim 1, wherein the antenna comprises a flat monopole antenna.

9. The implantable system of claim 1, wherein the antenna comprises a planar inverted-F antenna (PIFA).

10. The implantable system of claim 1, wherein the antenna is connected to the plurality of coils such that the plurality of coils serves as a ground plane for the antenna.

11. The implantable system of claim 10, wherein the antenna is connected to the plurality of coils through a single electrical connection.

12. The implantable system of claim 10, wherein the antenna is connected to the plurality of coils through a plurality of electrical connections.

13. The implantable system of claim 1, wherein, subsequent to being inserted into the cranial aneurysm, the antenna is positioned outside of a structure formed by the plurality of coils, and wherein the sensor and the communication circuitry are positioned at least partially inside the structure formed by the plurality of coils.

14. The implantable system of claim 13, wherein the plurality of coils are configured to form a substantially spherical shape when positioned within the cranial aneurysm.

15. The implantable system of claim 1, wherein the antenna, in an expanded configuration, is configured to form a substantially semi-spherical structure that facilitates occlusion of the flow of blood.

16. The implantable system of claim 1, wherein the antenna comprises first and second antennas configured to communicate in different frequency bands.

17. The implantable system of claim 16, wherein the first antenna, in an expanded configuration, is configured to form a first substantially semi-spherical structure that contours to one or more walls of the cranial aneurysm, and wherein the second antenna, in an expanded configuration, is configured to form a second substantially semi-spherical structure that facilitates occlusion of the flow of blood.

18. The implantable system of claim 17, wherein the first structure positioned opposite the second structure when the first and second antennas are in the expanded configuration.

19. The implantable system of claim 1, wherein the antenna is configured to transmit in a frequency range between 2400 MHz and 2483.5 MHz.

20. The implantable system of claim 1, wherein the antenna is configured to transmit and receive data over a distance of at least 12 centimeters.

21. The implantable system of claim 1, wherein the sensor comprises at least one of a blood flow sensor, a blood pressure sensor, a metabolic sensor, a glucose sensor, an oxygen sensor, or a pressure sensor.

22. The implantable system of claim 1, wherein the antenna comprises or is coated with a material that promotes clotting of blood.

23. The implantable system of claim 1, wherein the antenna comprises or is coated with a material that inhibits clotting of blood.

24. The implantable system of claim 1, further comprising a receiver configured to receive the data related to the property of blood flow detected by the sensor, wherein the receiver is configured to be supported by an article of clothing worn by a user in the upper torso or head.

25-64. (canceled)
